(19) United States
(12) Patent Application Publication
(10) Pub. No.: US 2025/0270535 A1
(43) Pub. Date: Aug. 28, 2025

(54) INCORPORATION OF UNNATURAL NUCLEOTIDES AND METHODS THEREOF

(71) Applicants: Synthorx, Inc., Bridgewater, NJ (US); The Scripps Research Institute, La Jolla, CA (US)

(72) Inventors: Jerod PTACIN, La Jolla, CA (US); Carolina CAFFARO, La Jolla, CA (US); Hans AERNI, La Jolla, CA (US); Yorke ZHANG, La Jolla, CA (US); Emil C. FISCHER, La Jolla, CA (US); Aaron W. FELDMAN, La Jolla, CA (US); Vivian T. DIEN, La Jolla, CA (US); Floyd E. ROMESBERG, La Jolla, CA (US)

(73) Assignees: Synthorx, Inc., Bridgewater, NJ (US); The Scripps Research Institute, La Jolla, CA (US)

(21) Appl. No.: 19/196,915

(22) Filed: May 2, 2025

Related U.S. Application Data
(62) Division of application No. 18/481,086, filed on Oct. 4, 2023, now Pat. No. 12,319,944.
Application No. 18/481,086 is a division of application No. 17/196,151, filed on Mar. 9, 2021, now Pat. No. 11,834,689.
Application No. 17/196,151 is a continuation of application No. 16/629,255, filed on Jan. 7, 2020, now abandoned.
(60) Provisional application No. 62/531,325, filed on Jul. 11, 2017.

(51) Int. Cl. C12N 9/00; C12N 15/113; C12N 15/90; C12P 21/02

(52) U.S. Cl. CPC C12N 9/93; C12N 15/113; C12N 15/90; C12P 21/02; C12Y 601/01026

(57) ABSTRACT

Disclosed herein are methods, compositions and kits for the synthesis of proteins which comprises unnatural amino acids that utilize a mutant tRNA.

### CROSS-REFERENCE

This application is a divisional of U.S. application Ser. No. 18/481,086, filed on Oct. 4, 2023, which is a divisional of U.S. application Ser. No. 17/196,151, filed on Mar. 9, 2021, which is a continuation of U.S. application Ser. No. 16/629,255, filed Jan. 7, 2020, which is the U.S. National Phase entry of International Application No. PCT/US2018/041509, filed Jul. 10, 2018, which claims the benefit of U.S. Provisional Application No. 62/531,325 filed on Jul. 11, 2017, each of which is incorporated herein by reference in its entirety.

### STATEMENT AS TO FEDERALLY SPONSORED RESEARCH

This invention was made with government support under Contract number GM060005 and GM118178 awarded by the National Institutes of Health and Contract number NSF/DGE-1346837 awarded by the National Science Foundation Graduate Research Fellowships. The government has certain rights in the invention.

### BACKGROUND OF THE INVENTION

Oligonucleotides and their applications have revolutionized biotechnology. However, the oligonucleotides including both DNA and RNA each includes only the four natural nucleotides of adenosine (A), guanosine (G), cytosine (C), thymine (T) for DNA, and the four natural nucleotides of adenosine (A), guanosine (G), cytosine (C), and uridine (U) for RNA, and which significantly restricts the potential functions and applications of the oligonucleotides.

The ability to sequence-specifically synthesize/amplify oligonucleotides (DNA or RNA) with polymerases, for example by PCR or isothermal amplification systems (e.g., transcription with T7 RNA polymerase), has revolutionized biotechnology. In addition to all of the potential applications in nanotechnology, this has enabled a diverse range of new technologies such as the in vitro evolution via SELEX (Systematic Evolution of Ligands by Exponential Enrichment) of RNA and DNA aptamers and enzymes. See, for example, Oliphant A R, Brandl C J & Struhl K (1989), Defining the sequence specificity of DNA-binding proteins by selecting binding sites from random-sequence oligonucleotides: analysis of yeast GCN4 proteins, Mol. Cell Biol., 9:2944-2949; Tuerk C & Gold L (1990), Systematic evolution of ligands by exponential enrichment: RNA ligands to bacteriophage T4 DNA polymerase, Science, 249:505-510; Ellington A D & Szostak J W (1990), In vitro selection of RNA molecules that bind specific ligands, Nature, 346:818-822.

In some aspects, these applications are restricted by the limited chemical/physical diversity present in the natural genetic alphabet (the four natural nucleotides A, C, G, and T in DNA, and the four natural nucleotides A, C, G, and U in RNA). Disclosed herein is an additional method of generating nucleic acids that contains an expanded genetic alphabet.

### SUMMARY OF THE INVENTION

Disclosed herein, in certain embodiments, are methods of producing a protein containing an unnatural amino acid, the method comprising: preparing a mutant tRNA wherein the mutant tRNA comprises a mutant anticodon sequence selected from Table 1 or 2; preparing a mutant mRNA wherein the mutant mRNA comprises a mutant codon sequence selected from Table 1 or 2; and synthesizing the protein containing an unnatural amino acid utilizing the mutant tRNA and the mutant mRNA. In some instances, the protein is synthesized in a cell-free translation system. In some instances, the protein is synthesized in a cell (semi-synthetic organism or SSO). In some instances, the semi-synthetic organism comprises a microorganism. In some instances, the semi-synthetic organism comprises a bacterium. In some instances, the semi-synthetic organism comprises an Escherichia coli. In some instances, the mutant anticodon of the mutant tRNA pairs with a mutant codon selected from Tables 1-3. In some instances, the unnatural amino acid comprises at least one unnatural nucleotide. In some instances, the unnatural nucleotide comprises an unnatural nucleobase. In some instances, the unnatural base of the unnatural nucleotide is selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifiuoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, O-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′: 4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladenine, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3) w, and 2,6-diaminopurine and those in which the purine or pyrimidine base is replaced with a heterocycle. In some instances, the unnatural nucleotide is selected from the group consisting of (only nucleobase portion shown, ribose and phosphate backbone omitted for clarity)

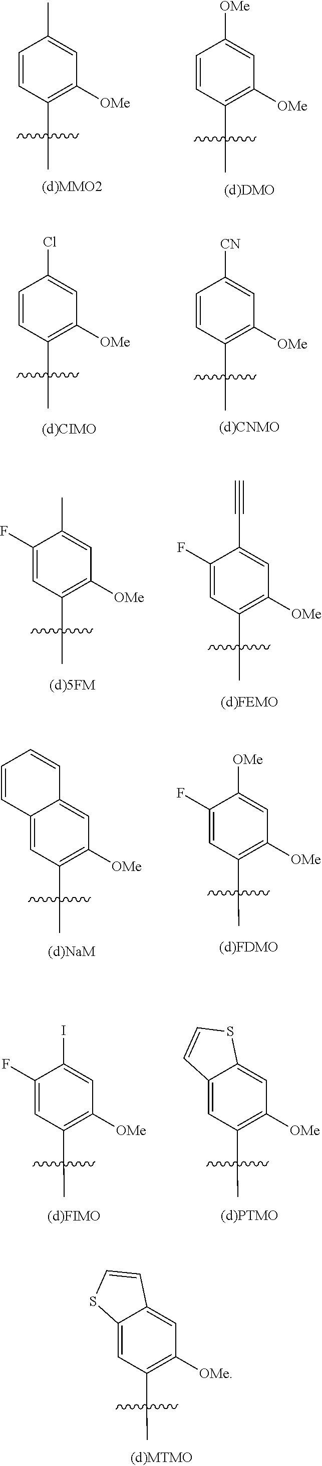

In some instances, the unnatural nucleotide is selected from the group consisting of (only nucleobase portion shown, ribose and phosphate backbone omitted for clarity)

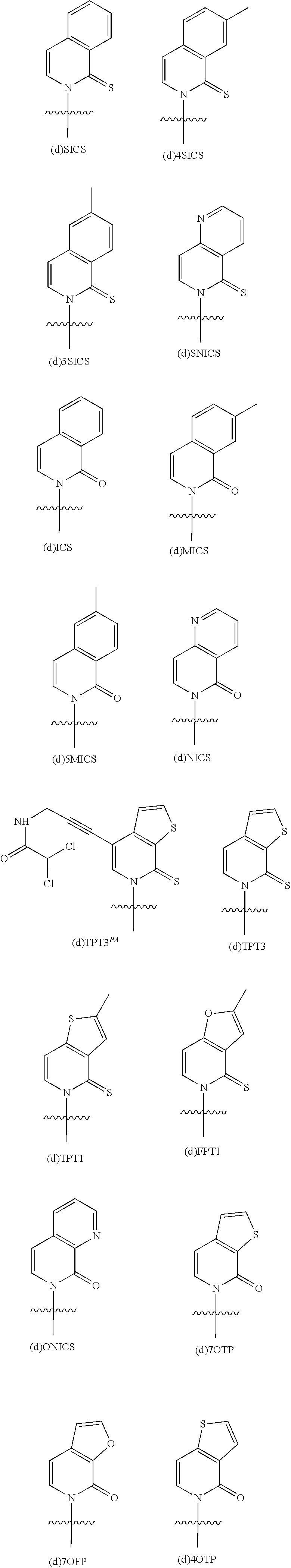

In some instances, the unnatural nucleotide further comprises an unnatural sugar moiety. In some instances, the unnatural sugar moiety of the unnatural nucleotide is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH_{3, 2}′-O (CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH2)ₙO]ₘCH₃, —OCH₂)ₙOCH₃, —O(CH₂)ₙNH₂, —O(CH₂)ₙCH₃, —O(CH₂)ₙ —ONH₂, and —O(CH₂)ₙON[(CH₂)ₙCH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some instances, the mutant anticodon or the mutant codon further comprises an unnatural backbone. In some instances, the mutant anticodon and the mutant codon further comprises an unnatural backbone. In some instances, the unnatural nucleotides are recognized by a DNA polymerase, an RNA polymerase, or a reverse transcriptase. In some instances, an unnatural nucleotide is incorporated by the RNA polymerase into the mRNA during transcription to generate a mutant mRNA containing a mutant codon. In some instances, an unnatural nucleotide is incorporated by the RNA polymerase into the tRNA during transcription to generate a mutant tRNA containing a mutant anticodon. In some instances, an unnatural nucleotide is incorporated by the RNA polymerase into the mRNA during transcription to generate a mutant mRNA. In some instances, an unnatural nucleotide is incorporated by the RNA polymerase into the tRNA during transcription to generate a mutant tRNA. In some instances, the mutant tRNA is charged with an unnatural amino acid residue. In some instances, a protein containing an unnatural amino acid is generated during translation utilizing the mutant tRNA and the mutant mRNA.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A illustrates the chemical structure of the dNaM-dTPT3 UBP and a natural dA-dT base pair.

FIG. 1B illustrates the gene cassette used to express sfGFP (AXC)¹⁵¹ and tRNA(GYT)^{Ser}. P_{T7} and T_{T7} denote the T7 RNAP promoter and terminator, respectively. In controls where sfGFP is expressed in the absence of serT, the sequence following the sfGFP T7 terminator is absent.

FIG. 1C illustrates a graph of fluorescence of cells expressing sfGFP and tRNA^{Ser} with the indicated position 151-codon and anticodon, respectively. Minus sign denotes the absence of serT in the expression cassette. t=0 corresponds to the addition of IPTG to induce expression of T7 RNAP and tRNA^{Ser} (if present); aTc was added at t=0.5 h to induce expression of sfGFP. AGT, natural Ser codon; TAG, amber stop codon; CTA amber suppressor anticodon. Data shown as mean ± s.d., n=4 cultures, each propagated from an individual colony.

FIG. 1D illustrates a graph of growth of cells expressing sfGFP and tRNA^{Ser} with the indicated position 151-codon and anticodon, respectively. Minus sign denotes the absence of serT in the expression cassette. t=0 corresponds to the addition of IPTG to induce expression of T7 RNAP and tRNA^{Ser} (if present); aTc was added at t=0.5 h to induce expression of sfGFP. AGT, natural Ser codon; TAG, amber stop codon; CTA amber suppressor anticodon. Data shown as mean ±s.d., n=4 cultures, each propagated from an individual colony.

FIG. 1E illustrates a Western blot of lysates (normalized by OD₆₀₀) from cells collected at the last time point shown in FIG. 1C and FIG. 1D, probed with an α-GFP antibody (N-terminal epitope).

FIG. 1F illustrates a graph of the relative abundance of the amino acids (indicated by their single letter codes in the figure legend) detected at position 151 of sfGFP purified from cells expressing sfGFP (AGT)¹⁵¹ or sfGFP (AXC)¹⁵¹ and tRNA^{Ser}(GYT), as determined by LC-MS/MS and precursor ion intensity based quantitation (amino acids detected at <0.1% (on average, for both codons) are not shown; see Methods for details and Table 4 for a complete list of amino acids detected). Data shown as mean with individual data points, n=4 purified sfGFP samples, each from a culture propagated from an individual colony and collected at the last time point shown in FIG. 1C and FIG. 1D.

FIG. 2A illustrates a graph of fluorescence of cells expressing sfGFP with the indicated position 151-codon, in the presence (+) or absence (−) of a tRNA^{Pyl} with a cognate anticodon, PylRS, or 20 mM PrK (N⁶-[(2-propynyloxy) carbonyl]-L-lysine) in the media. Fluorescence was determined at the last time point shown in FIG. 2B. Asterisk denotes the absence of tRNA^{Pyl} in cells expressing sfGFP (TAC) 151. TAC, natural Tyr codon; TAG, amber stop codon; n.d., not determined. Data shown as mean with individual data points, each propagated from an individual colony.

FIG. 2B illustrates a timecourse analysis of a subset of the data shown in FIG. 2A. Plus and minus signs denote the presence or absence, respectively, of 20 mM PrK in the media. t=0 corresponds to the addition of IPTG to induce expression of PylRS, T7 RNAP, and tRNA^{Pyl}; aTc was added at t=1 h to induce expression of sfGFP. Data shown as mean ±s.d., n=4 cultures, each propagated from an individual colony.

FIG. 2C illustrates Western blots of sfGFP purified from cells expressing sfGFP and tRNA^{Pyl} with the indicated position-151 codon and anticodon, respectively, with or without click conjugation of TAMRA and/or addition of 20 mM PrK to the media. tRNA^{Pyl} is absent (−) in cells expressing sfGFP (TAC) 151. sfGFP was purified from cultures collected at the last time point shown in FIG. 2B. Western blots were probed with an α-GFP antibody and imaged to detect both sfGFP and the conjugated TAMRA.

FIG. 2D illustrates a graph of the relative abundance of amino acids (indicated by their single letter codes in the figure legend) at position 151 of sfGFP purified from cells (collected at the last time point shown in FIG. 2B) expressing sfGFP (TAC)¹⁵¹ or sfGFP and tRNA^{Pyl} with the indicated position-151 codon and a cognate anticodon, respectively, as determined by LC-MS/MS and precursor ion intensity based quantitation (amino acids detected at <0.1% (on average, for all codons) are not shown; see Methods for details and Table 4 for a complete list of amino acids detected). Data shown as mean with individual data points, n=4 purified sfGFP samples, each from a culture propagated from an individual colony.

FIG. 3A illustrates a graph of fluorescence of cells expressing sfGFP (TAC)¹⁵¹ or sfGFP and tRNA^{pAzF} with the indicated position-151 codon and a cognate anticodon, respectively, in the presence (+) or absence (−) of 5 mM pAzF in the media. t=0 corresponds to the addition of IPTG to induce expression of pAzFRS, T7 RNAP, and tRNA^{pAzF}; aTc was added at t=0.5 h to induce expression of sfGFP. TAC, natural Tyr codon; TAG, amber stop codon. Data shown as mean ± s.d., n=4 cultures, each propagated from an individual colony. The fluorescence observed with sfGFP (AXC)¹⁵¹ in the absence of pAzF is attributed to charging of tRNA^{pAzF}(GYT) with a natural amino acid (likely Tyr).

FIG. 3B illustrates a Western blot of sfGFP purified from cells expressing sfGFP and tRNA^{pAzF} with the indicated position-151 codon and anticodon, respectively, with or without click conjugation of TAMRA and/or addition of 5 mM pAzF to the media. Where indicated, the minus sign denotes the absence of tRNA^{pAzF} in cells expressing sfGFP (TAC)¹⁵¹. sfGFP was purified from cultures collected at the last time point shown in FIG. 3A. Western blots were probed with an α-GFP antibody and imaged to detect both sfGFP and the conjugated TAMRA.

FIG. 4 illustrates fluorescence of cells expressing sfGFP with various codons at position 151. Cells carrying a sfGFP plasmid with the indicated position-151 codons were grown to an OD600˜0.5 and induced with IPTG and aTc. Fluorescence measurements were taken after 3 h of induction. Data shown as mean with individual data points, n=3 cultures split from a single colony and grown in parallel.

FIG. 5A illustrates decoding of the AXC codon with natural near-cognate anticodons, with a graph of fluorescence of cells expressing sfGFP (AXC)¹⁵¹ with or without tRNA^{Ser} with the indicated anticodon. Cells were induced as described in FIG. 1C and FIG. 1D and fluorescence measurements correspond to the last time point shown in FIG. 1C. Values for the GYT anticodon and in the absence of tRNA^{Ser} (-tRNA) correspond to the same values in FIG. 1c,d. Data shown as mean ± s.d., n=4 cultures, each propagated from an individual colony.

FIG. 5B illustrates decoding of the AXC codon with natural near-cognate anticodons, with a graph of growth of cells expressing sfGFP (AXC)¹⁵¹ with or without tRNA^{Ser} with the indicated anticodon. Cells were induced as described in FIG. 1C and FIG. 1D and fluorescence measurements correspond to the last time point shown in FIG. 1C. Values for the GYT anticodon and in the absence of tRNA^{Ser} (-tRNA) correspond to the same values in FIG. 1C and FIG. 1D. Data shown as mean ± s.d., n=4 cultures, each propagated from an individual colony.

FIG. 6A illustrates Western blots and growth of cells decoding AXC and GXC codons with tRNA^{Pyl}. Western blot of lysates (normalized by OD₆₀₀) from cells expressing sfGFP with the indicated position 151-codon, in the presence (+) or absence (−) of a tRNA^{Pyl} with a cognate anticodon, PylRS, or 20 mM PrK in the media. Blots were probed with an α-GFP antibody (N-terminal epitope). Cells were induced and collected at an equivalent time point as described in FIG. 2B.

FIG. 6B illustrates growth of cultures analyzed in FIG. 6A. The fold change in OD₆₀₀ between induction of sfGFP (t=1 h) and the final time point is greatest when all components necessary for aminoacylating tRNA^{Pyl} are present. Variations in the absolute value of OD₆₀₀ are due to small variations in cell density at the start of T7 RNAP (and if present tRNA^{Pyl}) induction (t=0). Data shown as mean ± s.d., n=4 cultures, each propagated from an individual colony.

FIG. 7A illustrates decoding AXC and GXC codons with tRNA^{Pyl} and cell growth as a function of added unnatural ribotriphosphates. Fluorescence of purified sfGFP (lower panel) from cells expressing sfGFP and tRNA^{Pyl} with the position 151-codon/anticodon indicated, in the presence (+) or absence (−) of each unnatural ribotriphosphate in the media, and with or without 20 mM PrK. Cells were induced as described in FIG. 2B and fluorescence measurements were taken at the end of induction (˜3.5 h), prior to collecting the cells and purifying the sfGFP protein for click conjugation of TAMRA and western blotting.

FIG. 7B illustrates a gel of decoding AXC and GXC codons with tRNA^{Pyl} as a function of added unnatural ribotriphosphates. Western blots were probed with an α-GFP antibody and imaged to detect both sfGFP and the conjugated TAMRA; all lanes correspond to sfGFP purified from cells grown with added PrK. Data shown as mean with individual data points, n=3 cultures, each propagated from an individual colony; n.d., not determined.

FIG. 7C, illustrates graphs of fluorescence and growth of cells expressing sfGFP (TAC)¹⁵¹ in the presence (+) or absence (−) of both unnatural deoxyribotriphosphates and each unnatural ribotriphosphate. t=0 corresponds to the addition of IPTG to induce expression of T7 RNAP; aTc was added at t=1 h to induce expression of sfGFP. Data shown as mean ±s.d., n=3 cultures, each propagated from an individual colony. At the concentrations used (see Methods), dNaMTP and dTPT3TP do not inhibit cell growth, whereas both unnatural ribotriphosphates, particularly TPT3TP, show some inhibition of growth.

FIG. 7D illustrates a graph of cell growth corresponding to the cultures with added PrK (20 mM) whose fluorescence is shown in FIG. 2B. Cells expressing sfGFP with natural codons were grown without any unnatural triphosphates, whereas cells expressing sfGFP with unnatural codons were grown with both unnatural deoxy- and ribotriphosphates. Data shown as mean ± s.d., n=4 cultures, each propagated from an individual colony.

FIG. 8A illustrates a gel of decoding AXC and GXC codons with tRNA^{Pyl} as a function of PrK concentration in the media. Western blots of sfGFP purified from cells expressing sfGFP and tRNA^{Pyl} with the indicated position-151 codon/anticodon, with click conjugation of TAMRA and the addition of PrK to the media at the indicated concentrations. sfGFP was induced and purified from cells collected as described in FIG. 2B. Western blots were probed with an α-GFP antibody and imaged to detect both sfGFP and the conjugated TAMRA.

FIG. 8B illustrates a graph of decoding AXC and GXC codons with tRNA^{Pyl} as a function of PrK concentration in the media. Fluorescence of cells (measured at the last time point shown in c) expressing sfGFP and tRNA^{Pyl} with the indicated position-151 codon and anticodon, respectively, as a function of PrK concentration in the media. Fluorescence values for 0 and 20 mM PrK are the same as the (−) and (+) PrK values, respectively, shown in FIG. 2B. Data shown as mean ± s.d., n=4 cultures, each propagated from an individual colony.

FIG. 8C illustrates a timecourse analysis of fluorescence. For clarity, only one representative culture is shown for each codon/anticodon pair and PrK concentration. Without being bound by theory, we attribute the low level of sfGFP produced in the absence of PrK to decoding by endogenous tRNAs and loss of UBP retention in sfGFP (Table 5). However, the relative amount of sfGFP that contains PrK (FIG. 8A) and absolute amount of sfGFP expressed (FIG. 8B and FIG. 8C) increased in a dose-dependent manner with increasing PrK in the media, ultimately resulting in nearly full incorporation of PrK, suggesting that endogenous read-through of the AXC and GXC codons can be efficiently suppressed with sufficient concentrations of charged PrK-tRNA^{Pyl}(GYT) or PrK-tRNA^{Pyl}(GYC).

FIG. 8D illustrates a timecourse analysis of cell growth at various concentrations of PrK for the experiment shown in FIG. 8C.

FIG. 9 illustrates cell growth of the cultures whose fluorescence is shown in FIG. 3A. Data shown as mean ± s.d., n=4 cultures, each propagated from an individual colony

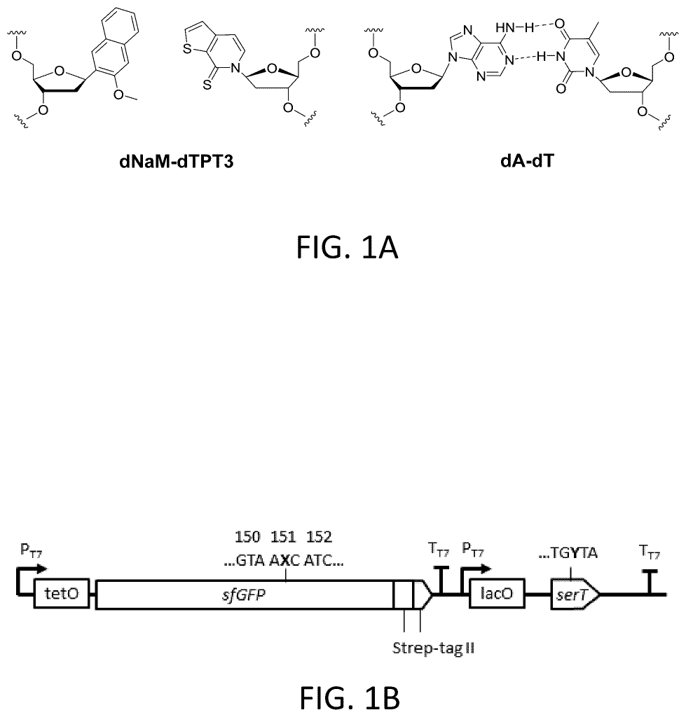

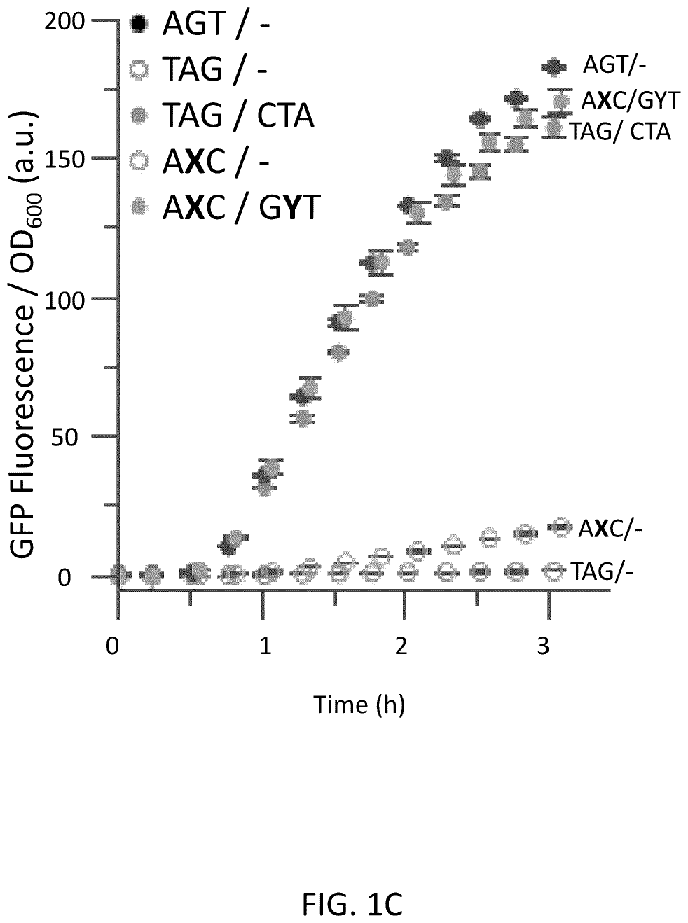

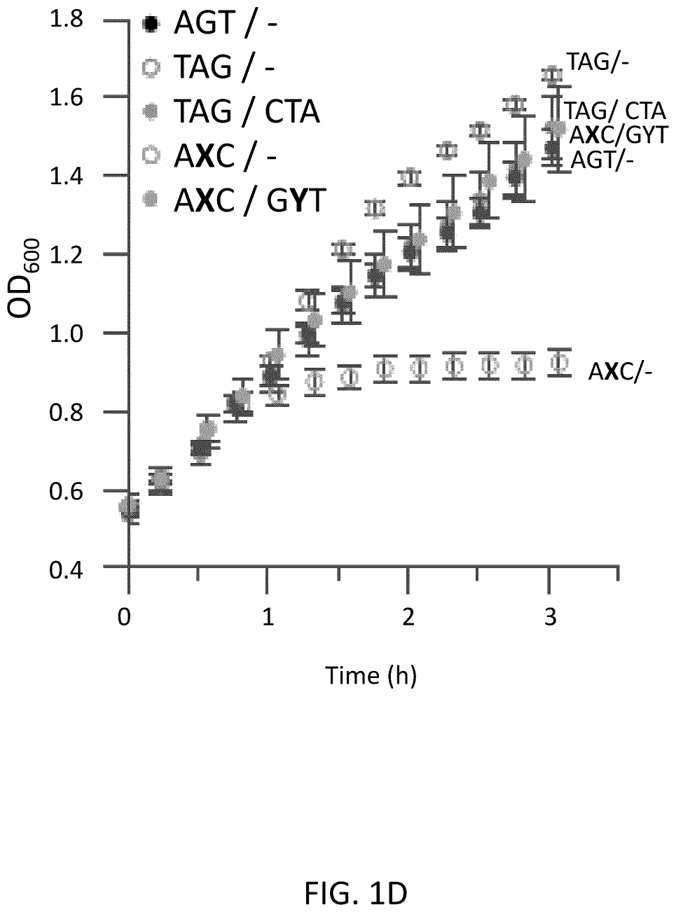

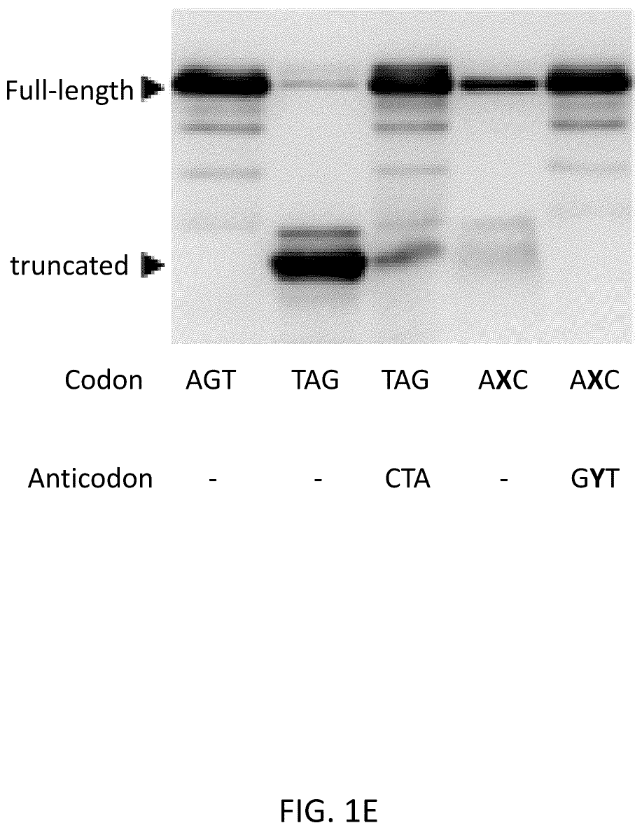

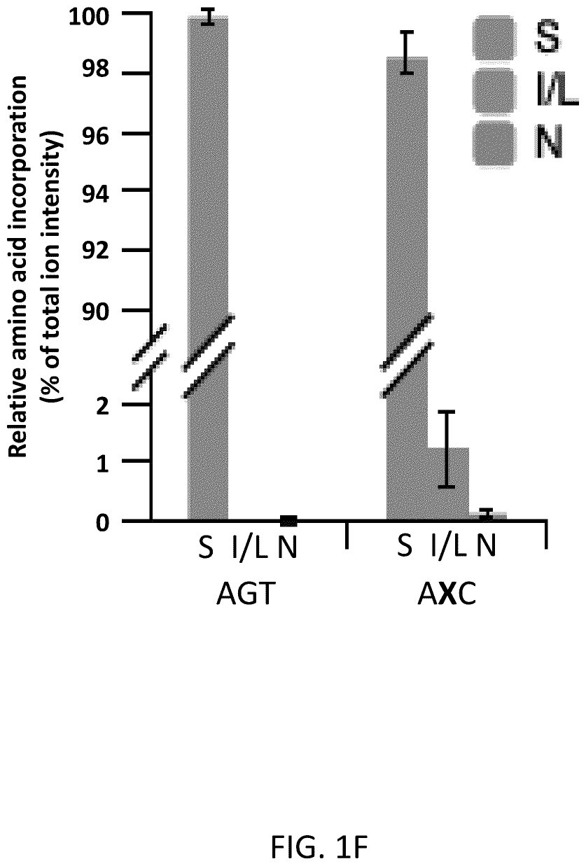

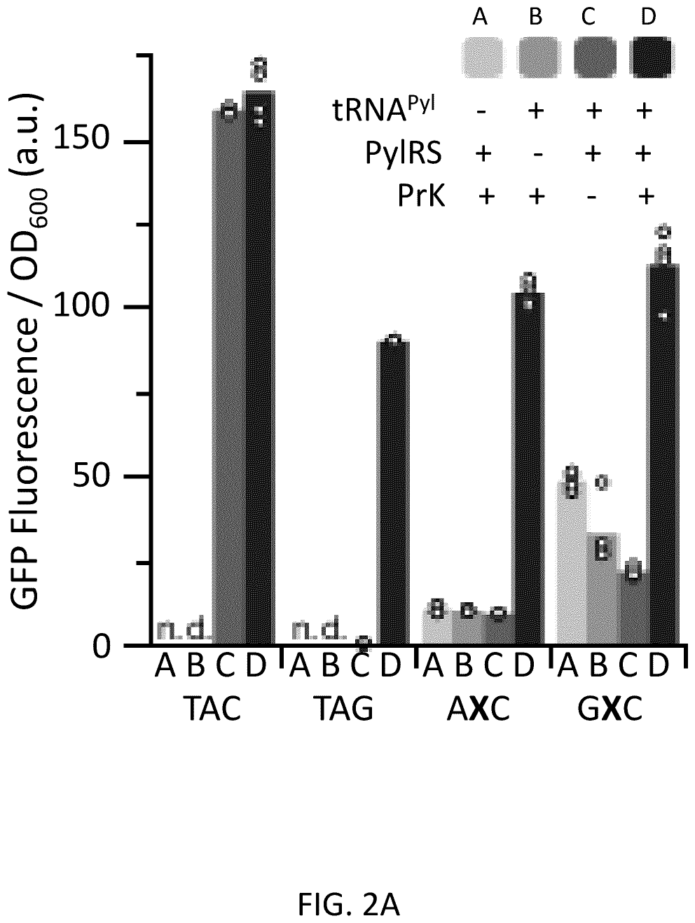

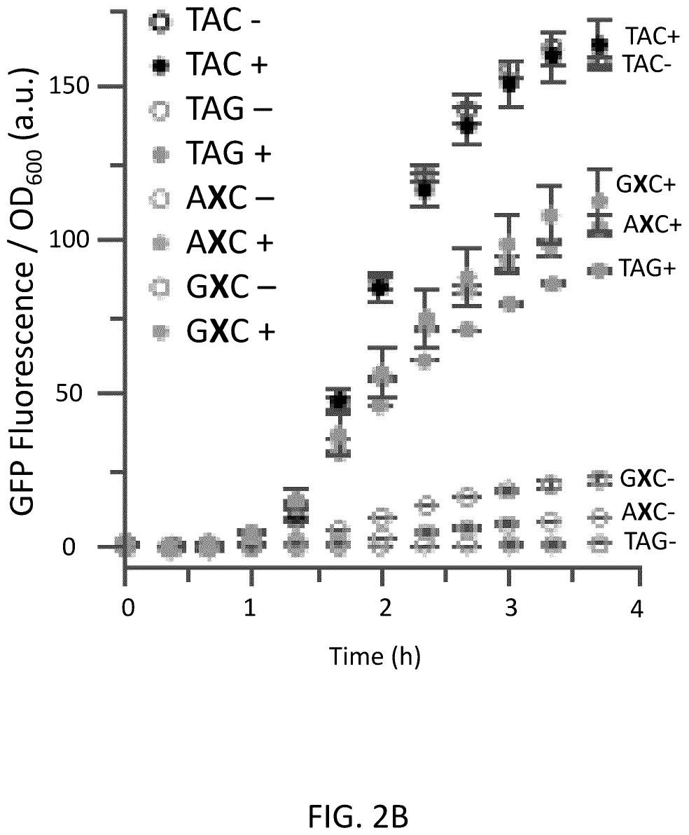

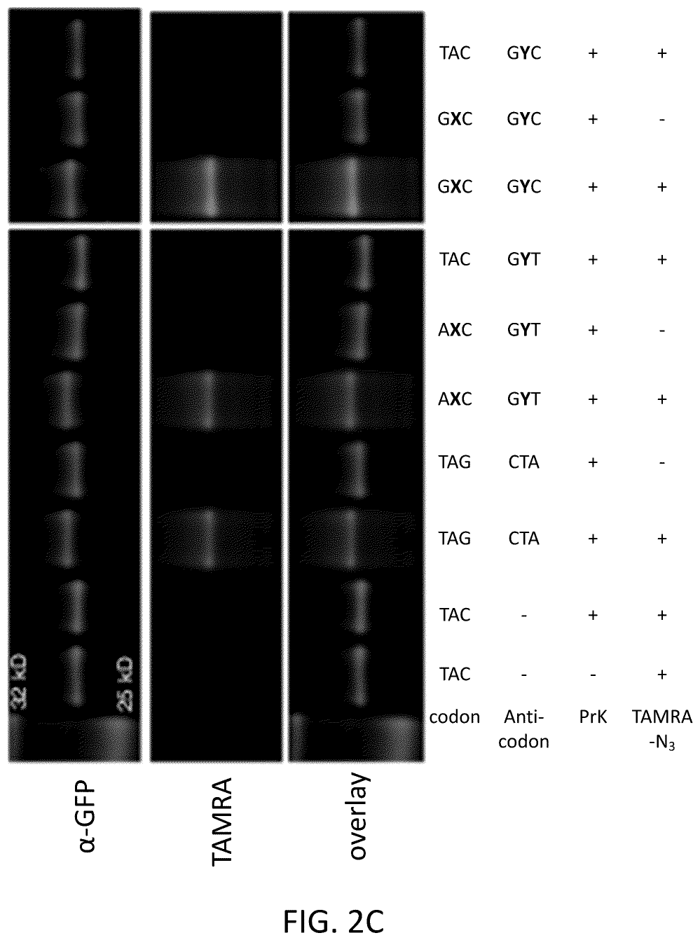

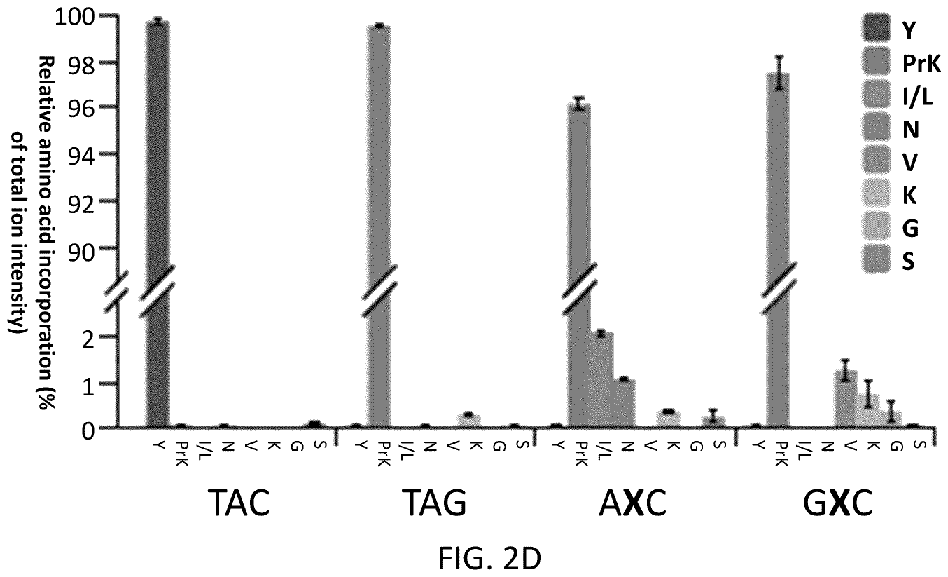

Table 4 | Relative abundance of amino acids at position 151 in sfGFP for experiments described in FIG. 1F and FIG. 2D. sfGFP purified from cells expressing sfGFP with or without tRNAs with the indicated position-151 codon and anticodon, respectively, were analyzed by LC-MS/MS. The extracted MS1 ion intensities for the reporter peptides LEYNFNSHNVX¹⁵¹ITADK (X=PrK or any identified natural amino acid except K or R) and LEYNFNSHNVX¹⁵¹ (if X=K or R) are expressed as a percentage of the sum of ion intensities for all observable reporter peptides. The table of values corresponds to the mean relative abundances and 95% CIs of all amino acids detected at position 151 of sfGFP, n=4 purified sfGFP samples, each from a culture propagated from an individual colony. Values <0.1% (on average, for the codons indicated in the respective figures) are excluded from the data presented in FIG. 1F and FIG. 2D.

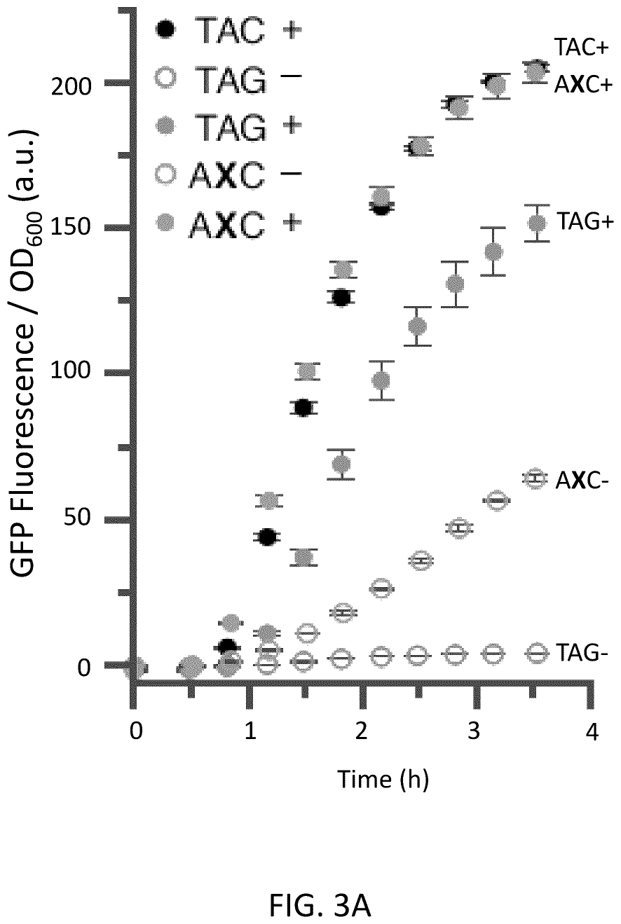

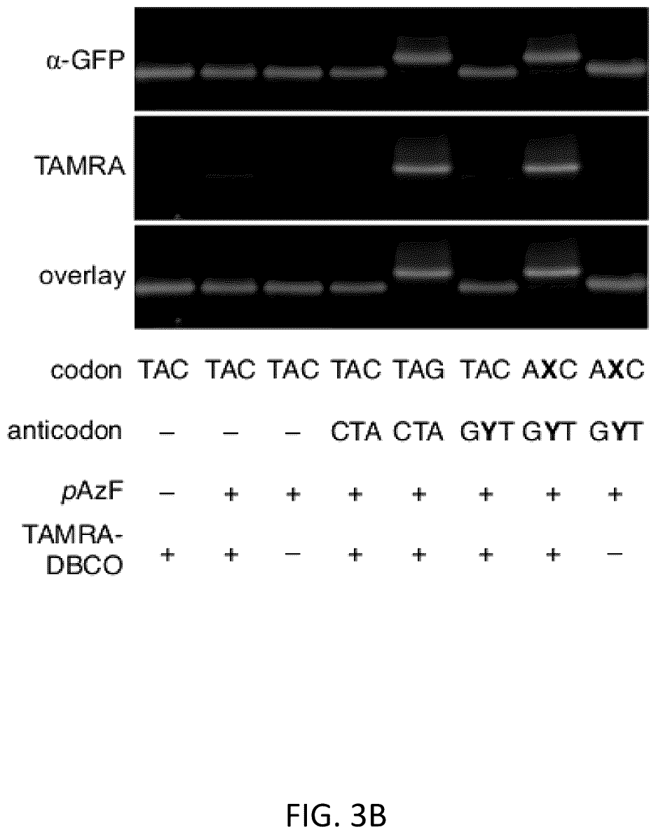

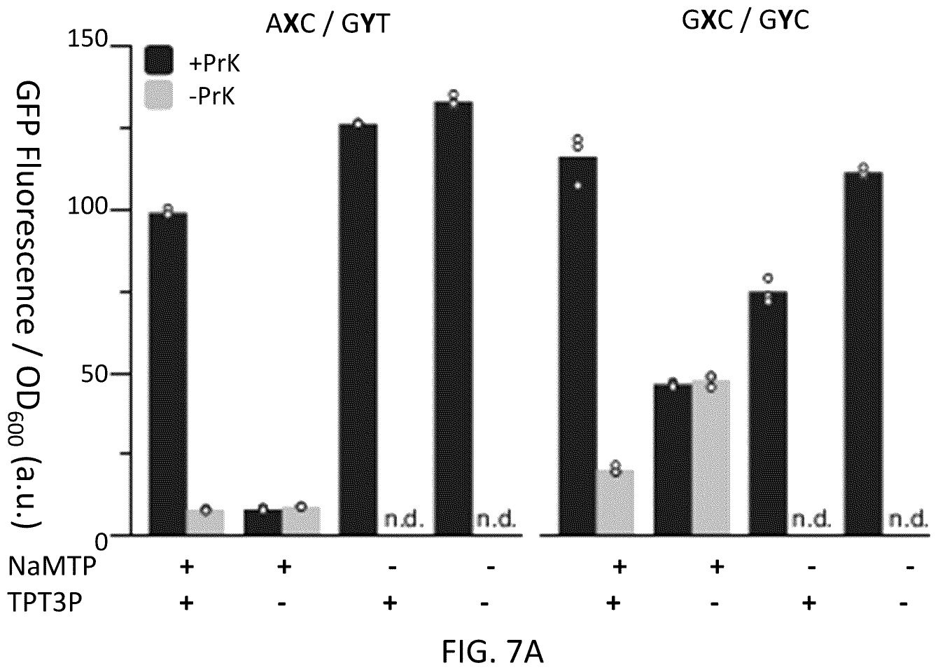

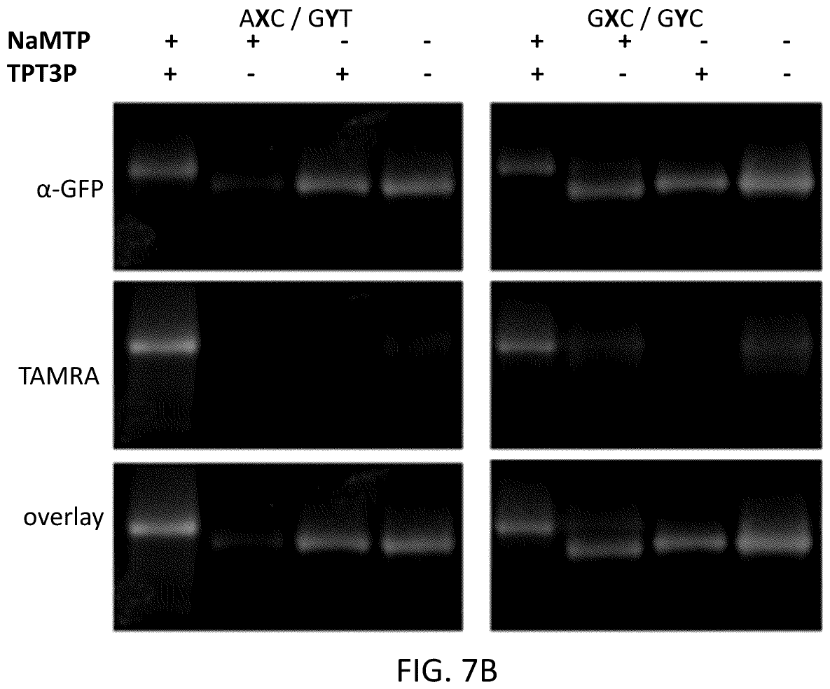

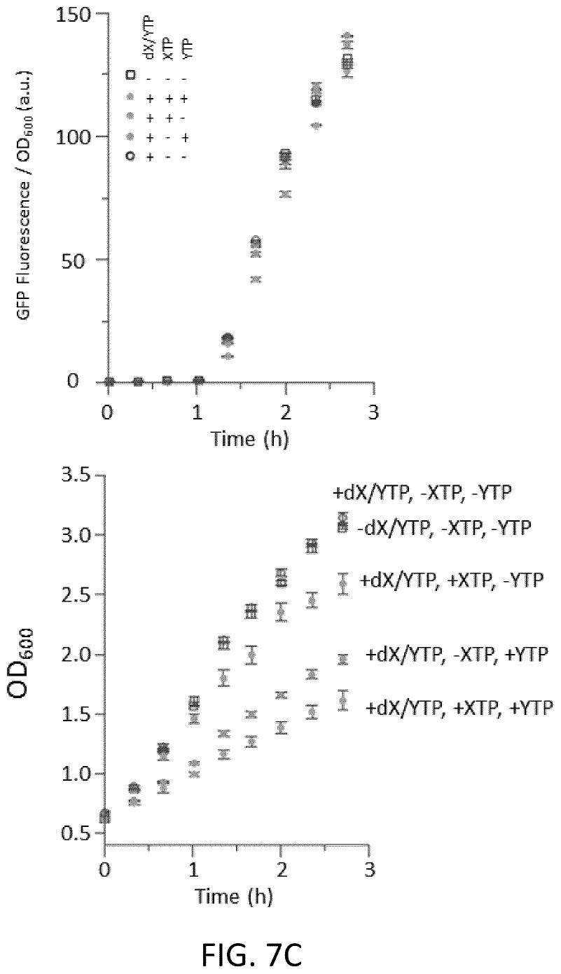

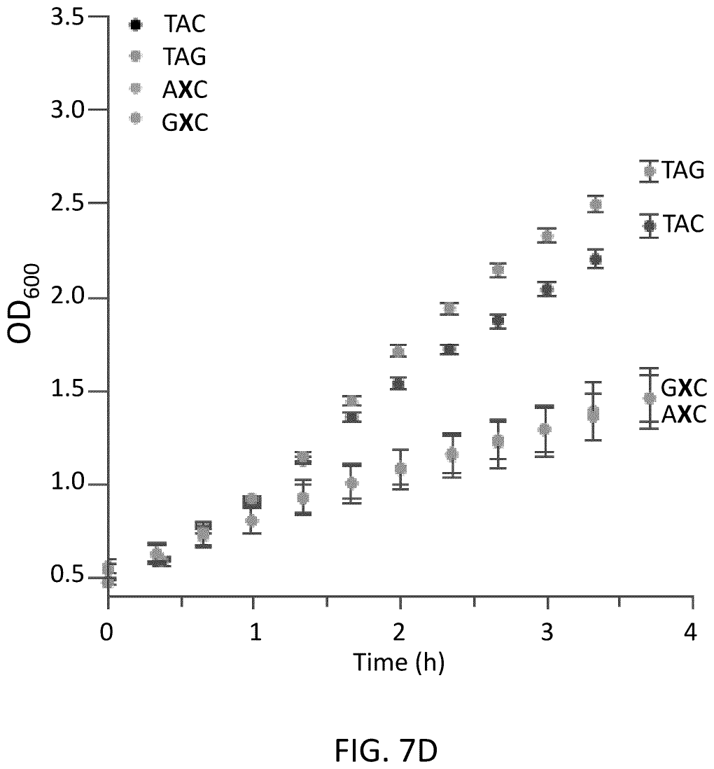

Table 5 | UBP retention. Retention of the UBP(s) in plasmids with the indicated position-151 codons of sfGFP and anticodons of the indicated tRNAs were determined for a time point prior to sfGFP induction and at the end of induction, as described in Methods. The reported values are the mean UBP retention over the course of the induction (calculated from the retentions at these two time points) ±95% CI, n=4 cultures, each propagated from an individual colony, except for values indicated with an asterisk, for which n=3. n/a, not applicable (because the relevant sequence is natural or absent). All plasmids were isolated from cultures grown in the presence of 20 mM PrK or 5 mM pAzF (except for Ser decoding experiments). SerRS indicates charging with the endogenous E. coli synthetase. Minus sign denotes the absence of PyIRS in cells with tRNA^{Pyl} or the absence of an ectopically expressed tRNA. Retentions in rows indicated with § correspond to cultures from which sfGFP was also purified and analyzed by LC-MS/MS and/or western blot of TAMRA-conjugated sfGFP (see FIG. 1F (Ser), FIG. 2D (PrK), and FIG. 3B (pAzF)); rows with an asterisk correspond to the cultures analyzed in FIGS. 7A-D. Despite the fact that all four unnatural triphosphates enter the cell through the same transporter and thus competitively inhibit one another's import, no differences in UBP retention were observed with the presence (+) or absence (−) of NaMTP and/or TPT3TP in the media. These data, and the requirement of both unnatural ribotriphosphates for high levels of sfGFP expression with high-fidelity PrK incorporation (FIGS. 7A-D), collectively demonstrate that the expression level of the PINTT2 transporter in YZ3 imports the requisite levels of unnatural triphosphates necessary to sustain UBP replication and transcription.

Table 6 | Yields of sfGFP protein expressed in Ser, Prk and pAzF incorporation experiments. Yields were calculated from the total amount of protein purified and the volume of culture used for purification (see Methods). Data are mean ± s.d. (n=4 sfGFP samples, each purified from a culture propagated from an individual colony) and were determined from the same cultures analyzed in FIG. 1F (for SerRS) and FIG. 2D (for PyIRS), as well as the cultures corresponding to the (+) pAzF samples in FIG. 3A (for pAzFRS). Yields of purified sfGFP are comparable to the mean total fluorescence (not normalized to OD₆₀₀) of the cultures from which they were purified. Fluorescence values correspond to the time point at which cells were collected for sfGFP purification; see FIG. 1C (Ser), FIG. 2B (PrK), and FIG. 3A (pAzF).

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

### Overview

The information of life is encoded by a four letter genetic alphabet, which is made possible by the selective formation of two base pairs: (d) G-(d) C and (d) A-dT/U. A third, unnatural base pair (UBP) formed between two synthetic nucleotides expands this system, thereby increasing the potential for information storage, and has profound academic and practical implications. Of the wide variety of synthetic nucleotide analogs that have been reported, several pair stably with one another within an otherwise natural DNA duplex, but are not recognized by polymerases, and indicating that the forces governing stable pairing in duplex DNA are not the same as those governing polymerase-mediated replication. As a result, different approaches have been taken to develop replicable UBPs, for example, UBPs that are designed to interact via complementary hydrogen bonding (H-bonding) patterns not employed by the natural nucleotides. Although the natural base pairs form via H-bonding, there is no reason to assume a priori that H-bonding is the only force sufficient to underlie the storage (or retrieval) of genetic information. For example, it has been demonstrated that the Klenow fragment of E. coli DNA polymerase I (Kf) pairs dA with the unnatural nucleotide dF, whose difluorotoluene nucleobase is a shape mimic of thymine that is incapable of significant H-bonding. This supports a “geometrical selection” mechanism of DNA replication and suggests that forces other than H-bonding also contribute to replication.

The development of UBPs that are replicated, transcribed, and translated into protein in vitro provide insights into the forces underlying the storage and retrieval of natural information, and also enable wide ranging applications in chemical and synthetic biology. However, the ultimate goal of many efforts to develop UBPs is their in vivo use as the foundation of a semi-synthetic organism (SSO)—an organism that stably stores and retrieves increased (un-natural or synthetic, meaning man made) information. Moreover, such an SSO has revolutionary practical applications, including for human health. Most notably, an SSO revolutionizes the growing field of protein therapeutics. However, compared to traditional small molecule therapeutics, protein therapeutics are severely limited in their molecular properties due to the finite chemical diversity available with the twenty natural amino acids.

We recently reported the creation of an E. coli SSO that by virtue of a nucleoside triphosphate transporter from Phaeodactylum tricornutum (PtNTT2), imports the requisite unnatural triphosphates from the media and then uses them to replicate a plasmid containing the UBP dNaM-dTPT3. We have since shown that DNA containing the UBP may be transcribed in the SSO by T7 RNA polymerase, and that when an unnatural nucleotide is incorporated into the codon of an mRNA, different tRNAs charged with ncAAs and containing the cognate unnatural nucleotide in their anticodon, can efficiently and selectively decode the unnatural codon. Because the UBP may be combined at different positions of different codons, this suggests that the UBP may be used to encode proteins with multiple, different ncAAs.

Disclosed herein in certain embodiments are methods, compositions, and kits for the synthesis of proteins which comprises unnatural amino acids that utilizes a mutant tRNA. In some instances, the protein is synthesized in a cell-free translation system. In some instances, the protein is synthesized in a cell or semi-synthetic organism (SSO). In some instances, the semi-synthetic organism comprises a microorganism. In some instances, the semi-synthetic organism comprises a bacterium. In some instances, the semi-synthetic organism comprises an Escherichia coli. In some instances, the mutant tRNA contains a mutant anticodon sequence. In some instances, the mutant anticodon sequence is an anticodon sequence illustrated in Table 1. In some instances, the mutant anticodon sequence is an anticodon sequence illustrated in Table 2. In some instances, the mutant anticodon sequence is an anticodon sequence illustrated in Table 3.

TABLE 1

| | | |
|---|---|---|
| GGY | GYG | YGG |
| GAY | GYA | YGA |
| GCY | GYC | YGC |
| GUY | GYU | YGU |
| CAY | CYA | YCA |
| CGY | CYG | YCG |
| CUY | CYU | YCU |
| CCY | CYC | YCC |
| AAY | AYA | YAA |
| AGY | AYG | YAG |
| ACY | AYC | YAC |
| AUY | AYU | YAU |
| UUY | UYU | YUU |
| UAY | UYA | YUA |
| UGY | UYG | YUG |
| UCY | UYC | YUC |
| GYY | YGY | YYG |
| CYY | YCY | YYC |
| AYY | YAY | YYA |
| UYY | YUY | YYU |
| YYY | | |

TABLE 2

| | | |
|---|---|---|
| GGX | GXG | XGG |
| GAX | GXA | XGA |
| GCX | GXC | XGC |
| GUX | GXU | XGU |
| CAX | CXA | XCA |
| CGX | CXG | XCG |
| CUX | CXU | XCU |
| CCX | CXC | XCC |
| AAX | AXA | XAA |
| AGX | AXG | XAG |
| ACX | AXC | XAC |
| AUX | AXU | XAU |
| UUX | UXU | XUU |
| UAX | UXA | XUA |
| UGX | UXG | XUG |
| UCX | UXC | XUC |
| GXX | XGX | XXG |
| CXX | XCX | XXC |
| AXX | XAX | XXA |
| UXX | XUX | XXU |
| XXX | | |

TABLE 3

| | | |
|---|---|---|
| GXY | GYX | XYG |
| YXG | XGY | YGX |
| AXY | AYX | XYA |
| YXA | XAY | YAX |
| CXY | CYX | XYC |
| YXC | XCY | YCX |
| UXY | UYX | XYU |
| YXU | XUY | YUX |
| XYY | XXY | YXX |
| YXX | YXY | XYX |

In some instances, the mutant anticodon of the mutant tRNA pairs with a mutant codon. In some embodiments, the mutant codon is a mutant codon illustrated in Table 1. In some embodiments, the mutant codon is a mutant codon illustrated in Table 2. In some embodiments, the mutant codon is a mutant codon illustrated in Table 3.

In some embodiments, the Y and X illustrated in Table 1, Table 2, and Table 3 represent unnatural bases of the unnatural nucleotide. In some embodiments, the unnatural base is selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifiuoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, O-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine ([5,4-b][1,4] benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido [5,4-b][1,4] benzoxazin-2(3H)-one), carbazole cytidine (2H-pyrimido[4,5-b]indol-2-one), pyridoindole cytidine (H-pyrido[3′,2′: 4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladenine, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5oxacetic acid methylester, uracil-5-oxacetic acid, 5-methyl-2-thiouracil, 3-(3-amino-3-N-2-carboxypropyl) uracil, (acp3) w, and 2,6-diaminopurine and those in which the purine or pyrimidine base is replaced with a heterocycle.

In some instances, the unnatural nucleotide is selected from the group consisting of (only nucleobase portion shown, ribose and phosphate backbone omitted for clarity)

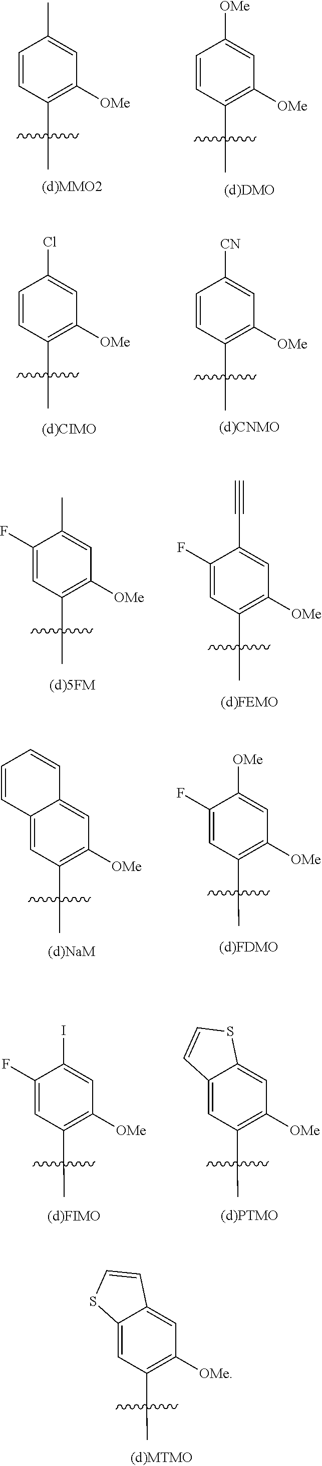

In some instances, the unnatural nucleotide is selected from the group consisting of (only nucleobase portion shown, ribose and phosphate backbone omitted for clarity)

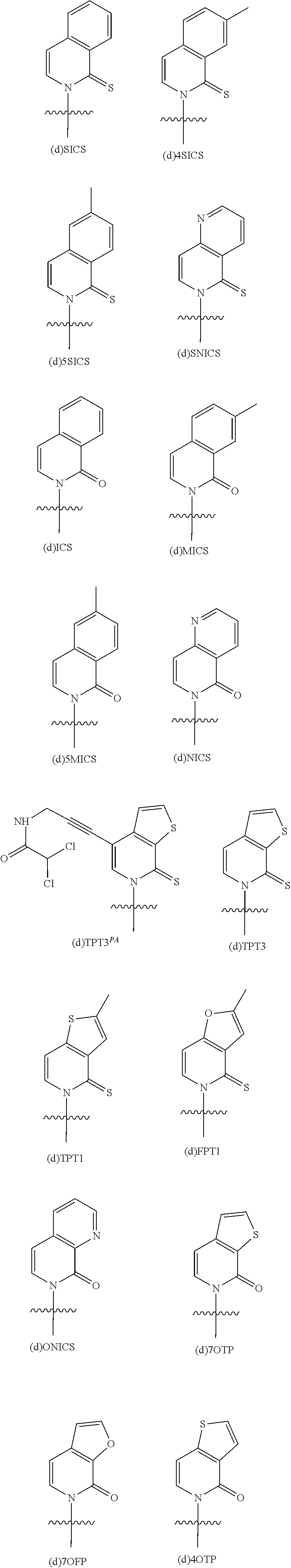

In some instances, the unnatural nucleotide further comprises an unnatural sugar moiety. In some instances, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O (CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH2)ₙ O]ₘCH₃, —O(CH₂)nOCH₃, —O(CH₂)n NH₂, —O(CH₂)n CH₃, —O(CH₂)n —ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.

In some instances, the mutant anticodon or the mutant codon further comprises an unnatural backbone. In some instances, the mutant anticodon further comprises an unnatural backbone. In some instances, the mutant codon further comprises an unnatural backbone. In some instances, the unnatural backbone is selected from the group consisting of a phosphorothioate, chiral phosphorothioate, phosphorodithioate, phosphotriester, aminoalkylphosphotriester, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates.

In some instances, the unnatural nucleotides are recognized by a polymerase. In some instances, the polymerase is a DNA polymerase, an RNA polymerase, or a reverse transcriptase. In some instances, the polymerase comprises Φ29, B103, GA-1, PZA, Φ15, BS32, M2Y, Nf, G1, Cp-1, PRD1, PZE, SF5, Cp-5, Cp-7, PR4, PR5, PR722, L17, ThermoSequenase®, 9°Nm™, Therminator™ DNA polymerase, Tne, Tma, TfI, Tth, TIi, Stoffel fragment, Vent™ and Deep Vent™ DNA polymerase, KOD DNA polymerase, Tgo, JDF-3, Pfu, Taq, T7 DNA polymerase, T7 RNA polymerase, PGB-D, UlTma DNA polymerase, E. coli DNA polymerase I, E. coli DNA polymerase III, archaeal DP1I/DP2 DNA polymerase II, 9°N DNA Polymerase, Taq DNA polymerase, Phusion® DNA polymerase, Pfu DNA polymerase, SP6 RNA polymerase, RB69 DNA polymerase, Avian Myeloblastosis Virus (AMV) reverse transcriptase, Moloney Murine Leukemia Virus (MMLV) reverse transcriptase, SuperScript® II reverse transcriptase, and SuperScript® III reverse transcriptase.

In some instances, the polymerase is DNA polymerase 1-Klenow fragment, Vent polymerase, Phusion® DNA polymerase, KOD DNA polymerase, Taq polymerase, T7 DNA polymerase, T7 RNA polymerase, Therminator™ DNA polymerase, POLB polymerase, SP6 RNA polymerase, E. coli DNA polymerase I, E. coli DNA polymerase III, Avian Myeloblastosis Virus (AMV) reverse transcriptase, Moloney Murine Leukemia Virus (MMLV) reverse transcriptase, SuperScript® II reverse transcriptase, or SuperScript® III reverse transcriptase.

In some instances, an unnatural nucleotide is incorporated by the polymerase into the mRNA during transcription to generate a mutant mRNA containing a mutant codon. In some instances, an unnatural nucleotide is incorporated by the polymerase into the mRNA during transcription to generate a mutant mRNA.

In some instances, an unnatural nucleotide is incorporated by the polymerase into the tRNA during transcription to generate a mutant tRNA containing a mutant anticodon. In some instances, an unnatural nucleotide is incorporated by the polymerase into the tRNA during transcription to generate a mutant tRNA.

In some instances, the mutant tRNA represents an unnatural amino acid residue. In some instances, an unnatural amino acid residue is a non-natural amino acid such as those described in Liu C. C., Schultz, P. G. Annu. Rev. Biochem. 2010, 79, 413.

In some instances, a protein containing an unnatural amino acid is generated during translation utilizing the mutant tRNA and the mutant mRNA. In some instances, the protein containing an unnatural amino acid is generated under a cell free translation system. In some instances, the protein is synthesized in a cell or semi-synthetic organism (SSO). In some instances, the semi-synthetic organism comprises a microorganism. In some instances, the semi-synthetic organism comprises a bacterium. In some instances, the semi-synthetic organism comprises an Escherichia coli.

### Nucleic Acids

A nucleic acid (e.g., also referred to herein as target nucleic acid, target nucleotide sequence, nucleic acid sequence of interest or nucleic acid region of interest) can be from any source or composition, such as DNA, cDNA, gDNA (genomic DNA), RNA, siRNA (short inhibitory RNA), RNAi, tRNA or mRNA, for example, and can be in any form (e.g., linear, circular, supercoiled, single-stranded, double-stranded, and the like). Nucleic acids can comprise nucleotides, nucleosides, or polynucleotides. Nucleic acids can comprise natural and unnatural nucleic acids. A nucleic acid can also comprise unnatural nucleic acids, such as DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Exemplary natural nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP. Exemplary natural deoxyribonucleotides include dATP, dTTP, dCTP, dGTP, dADP, dTDP, dCDP, dGDP, dAMP, dTMP, dCMP, and dGMP. Exemplary natural ribonucleotides include ATP, UTP, CTP, GTP, ADP, UDP, CDP, GDP, AMP, UMP, CMP, and GMP. For RNA, the uracil base is uridine. A nucleic acid sometimes is a vector, plasmid, phage, autonomously replicating sequence (ARS), centromere, artificial chromosome, yeast artificial chromosome (e.g., YAC) or other nucleic acid able to replicate or be replicated. An unnatural nucleic acid can be a nucleic acid analogue.

### Unnatural Nucleic Acids

A nucleotide analog, or unnatural nucleotide, comprises a nucleotide which contains some type of modification to either the base, sugar, or phosphate moieties. A modification can comprise a chemical modification. Modifications may be, for example, of the 3′OH or 5′OH group, of the backbone, of the sugar component, or of the nucleotide base. Modifications may include addition of non-naturally occurring linker molecules and/or of interstrand or intrastrand cross links. In one aspect, the modified nucleic acid comprises modification of one or more of the 3′OH or 5′OH group, the backbone, the sugar component, or the nucleotide base, and/or addition of non-naturally occurring linker molecules. In one aspect a modified backbone comprises a backbone other than a phosphodiester backbone. In one aspect a modified sugar comprises a sugar other than deoxyribose (in modified DNA) or other than ribose (modified RNA). In one aspect a modified base comprises a base other than adenine, guanine, cytosine or thymine (in modified DNA) or a base other than adenine, guanine, cytosine or uracil (in modified RNA).

The nucleic acid may comprise at least one modified base. Modifications to the base moiety would include natural and synthetic modifications of A, C, G, and T/U as well as different purine or pyrimidine bases. In some embodiments, a modification is to a modified form of adenine, guanine cytosine or thymine (in modified DNA) or a modified form of adenine, guanine cytosine or uracil (modified RNA).

A modified base of a unnatural nucleic acid includes but is not limited to uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CI¼) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4] benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4] benzoxazin-2(3H)-one), carbazole cytidine (2H-pyrimido [4,5-b]indol-2-one), pyridoindole cytidine (H-pyrido [3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone,, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594, 121; 5,596,091; 5,614,617; 5,645,985; 5,681,941; 5,750,692; 5,763,588; 5,830,653 and 6,005,096; WO 99/62923; Kandimalla et al. (2001) Bioorg. Med. Chem. 9:807-813; The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; and Sanghvi, Y. S., Chapter 15, Antisense Research and Applications, Crooke, S. T. and Lebleu, B., Eds., CRC Press, 1993, 273-288. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993.

Unnatural nucleic acids comprising various heterocyclic bases and various sugar moieties (and sugar analogs) are available in the art, and the nucleic acid can include one or several heterocyclic bases other than the principal five base components of naturally-occurring nucleic acids. For example, the heterocyclic base may include uracil-5-yl, cytosin-5-yl, adenin-7-yl, adenin-8-yl, guanin-7-yl, guanin-8-yl, 4-aminopyrrolo [2.3-d] pyrimidin-5-yl, 2-amino-4-oxopyrolo [2, 3-d] pyrimidin-5-yl, 2-amino-4-oxopyrrolo [2.3-d] pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, 3,687,808; 4,469,863; 4,476,301; 5,023,243; 5,177,196; 5,188,897; 5,264,423; 5,276,019; 5,278,302; 5,286,717; 5,321, 131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Unnatural nucleic acids can include 2′,3′-dideoxy-2′,3′-didehydro-nucleosides (PCT/US2002/006460), 5′-substituted DNA and RNA derivatives (PCT/US2011/033961; Saha et al, J. Org Chem., 1995, 60, 788-789; Wang et al, Bioorganic & Medicinal Chemistry Letters, 1999, 9, 885-890; and Mikhailov et al, Nucleosides & Nucleotides, 1991, 10 (1-3), 339-343; Leonid et al, 1995, 14 (3-5), 901-905; and Eppacher et al, Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al, Nucleosides Nucleotides & Nucleic Acids, 2004, 23 (1 & 2), 317-337).

Unnatural nucleic acids can include modifications at the 5′-position and the 2′-position of the sugar ring (PCT/US94/02993), such as 5′-CH₂ substituted 2′-O-protected nucleosides (Wu et al., Helvetica Chimica Acta, 2000, 83, 1127-1143 and Wu et al. Bioconjugate Chem. 1999, 10, 921-924). Unnatural nucleic acids can include amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al, Synlett, 1997, 1287-1290). Unnatural nucleic acids can include 2′-substituted 5′-CH₂ (or O) modified nucleosides (PCT/US92/01020). Unnatural nucleic acids can include 5′methylenephosphonate DNA and RNA monomers, and dimers (Bohringer et al, Tet. Lett., 1993, 34, 2723-2726; Collingwood et al, Synlett, 1995, 7, 703-705; and Hutter et al, Helvetica Chimica Acta, 2002, 85, 2777-2806). Unnatural nucleic acids can include 5′-phosphonate monomers having a 2′-substitution (US 2006/0074035) and other modified 5′-phosphonate monomers (WO 97/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and or 6′ position (Chen et al, Phosphorus, Sulfur and Silicon, 2002, 777, 1783-1786; Jung et al, Bioorg. Med. Chem., 2000, 8, 2501-2509, Gallier et al, Eur. J. Org. Chem., 2007, 925-933 and Hampton et al, J. Med. Chem., 1976, 19 (8), 1029-1033). Unnatural nucleic acids can include 5′-phosphonate deoxyribonucleoside monomers and dimers having a 5′-phosphate group (Nawrot et al, Oligonucleotides, 2006, 16 (1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al, Synlett, 2001, 4, 467-472; Kappler et al, J. Med. Chem., 1986, 29, 1030-1038 and J. Med. Chem., 1982, 25, 1179-1184; Vrudhula et al, J. Med. Chem., 1987, 30, 888-894; Hampton et al, J. Med. Chem., 1976, 19, 1371-1377; Geze et al, J. Am. Chem. Soc, 1983, 105 (26), 7638-7640 and Hampton et al, J. Am. Chem. Soc, 1973, 95 (13), 4404-4414)

Unnatural nucleic acids can also include modifications of the sugar moiety. Nucleic acids of the invention can optionally contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substitutent groups (including 5′ and/or 2′ substituent groups; bridging of two ring atoms to form bicyclic nucleic acids (BNA); replacement of the ribosyl ring oxygen atom with S, N (R), or C (R₁) (R₂) (R═H, C₁-C₁₂ alkyl or a protecting group); and combinations thereof. Examples of chemically modified sugars can be found in WO 2008/101157, US 2005/0130923, and WO 2007/134181.

A modified nucleic acid may comprise modified sugars or sugar analogs. Thus, in addition to ribose and deoxyribose, the sugar moiety can be pentose, deoxypentose, hexose, deoxyhexose, glucose, arabinose, xylose, lyxose, and a sugar “analog” cyclopentyl group. The sugar can be in pyranosyl or in a furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy-or amino-RNA/DNA chimeras. For example, a sugar modification may include, 2′-O-methyl-uridine and 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include but are not limited to the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙ O]ₘ CH₃, —O(CH₂)ₙOCH₃, —O(CH₂)ₙ NH₂, —O(CH₂)ₙ CH₃, —O(CH₂)ₙ —ONH₂, and —O(CH₂)ₙON[(CH₂)n CH₃)J₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,71 1; 5,552,540; 5,587,469; 5,594, 121, 5,596,091; 5,614,617; 5,681,941; and 5,700,920, each of which is herein incorporated by reference in its entirety, which detail and describe a range of base modifications. Each of these patents is herein incorporated by reference.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—C C₁₀ alkyl, OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids of the present invention include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)-0-2′,and analogs thereof (see, U.S. Pat. No. 7,399,845, issued on Jul. 15, 2008); 4′—C(CH₃) (CH₃)-0-2′and analogs thereof, (see WO2009/006478, WO2008/150729, US2004/0171570, U.S. Pat. No. 7,427,672, Chattopadhyaya, et al, J. Org. Chem.,2 09, 74, 118-134), and WO 2008/154401, published on Dec. 8, 2008). Also see, for example: Singh et al., Chem. Commun., 1998, 4, 455-456; Koshkin et al, Tetrahedron, 1998, 54, 3607-3630; Wahlestedt et al., Proc. Natl. Acad. Sci. U. S. A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al, J. Am. Chem. Soc, 129 (26) 8362-8379 (Jul. 4, 2007); Elayadi et al, Curr. Opinion Invens. Drugs, 2001, 2, 558-561; Braasch et al, Chem. Biol, 2001, 8, 1-7; Oram et al, Curr. Opinion Mol Ther., 2001, 3, 239-243; U.S. Pat. Nos. 7,053,207, 6,268,490, 6,770,748, 6,794,499, 7,034,133, 6,525,191, 6,670,461, and 7,399,845;; International applications WO 2004/106356, WO 1994/14226, WO 2005/021570, and WO 2007/134181; U.S. Patent Publication Nos. US2004/0171570, US2007/0287831, and US2008/0039618; U.S. patents Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT US2008/066154, and PCT US2008/068922, PCT/DK98/00393; and U.S. Pat. Nos. 4,849,513; 5,015,733; 5,118,800; and 5,118,802.

In certain embodiments, nucleic acids can comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)2-O—); and N,N*-dimethylhydrazine (—CH₂—N(CH₃)—N(CH₃)—). In certain embodiments, inter nucleic acids linkages having a chiral atom can be prepared a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

A phosphorous derivative (or modified phosphate group) can be attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al. (1996) Nucleic Acids Res. 24:1841-1848; Chaturvedi et al. (1996) Nucleic Acids Res. 24:2318-2323; and Schultz et al. (1996) Nucleic Acids Res. 24:2966-2973; Matteucci (1997) “Oligonucleotide Analogs: an Overview” in Oligonucleotides as Therapeutic Agents, (D J. Chadwick and G. Cardew, ed.) John Wiley and Sons, New York, NY; (Zon (1993) “Oligonucleoside Phosphorothioates” in Protocols for Oligonucleotides and Analogs, Synthesis and Properties (Agrawal, ed.) Humana Press, pp. 165-190); (Miller et al. (1971) JACS 93:6657-6665); (Jager et al. (1988) Biochem. 27:7247-7246), (Nelson et al. (1997) JOC 62:7278-7287) (U.S. Pat. No. 5,453,496); Micklefield, J. 2001, Current Medicinal Chemistry 8:1157-1179.

Backbone modification may comprise replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene (methylimino) linkages; formacetal and thioformacetal linkages; backbones containing sulfonyl groups; morpholino oligos; peptide nucleic acids (PNA); and positively charged deoxyribonucleic guanidine (DNG) oligos, Micklefield, J. 2001, Current Medicinal Chemistry 8:1157-1179. A modified nucleic acid may comprise a chimeric or mixed backbone comprising one or more modifications, e.g. a combination of phosphate linkages such as a combination of phosphodiester and phosphorothioate linkages.

Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216, 141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference. It is also understood in a nucleotide substitute that both the sugar and the phosphate moieties of the nucleotide can be replaced, by for example an amide type linkage (aminoethylglycine) (PNA). U.S. Pat. No. 5,539,082; 5,714,331; and 5,719,262 teach how to make and use PNA molecules, each of which is herein incorporated by reference. (See also Nielsen et al., Science, 1991, 254, 1497-1500). Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al, FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5, 118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

### Polymerase

A particularly useful function of a polymerase is to catalyze the polymerization of a nucleic acid strand using an existing nucleic acid as a template. Other functions that are useful are described elsewhere herein. Examples of useful polymerases include DNA polymerases and RNA polymerases.

The ability to improve specificity, processivity, or other features of polymerases unnatural nucleic acids would be highly desirable in a variety of contexts where, e.g., unnatural nucleic acid incorporation is desired, including amplification, sequencing, labeling, detection, cloning, and many others. The present invention provides polymerases with modified properties for unnatural nucleic acids, methods of making such polymerases, methods of using such polymerases, and many other features that will become apparent upon a complete review of the following.

In some instances, disclosed herein includes polymerases that incorporate unnatural nucleic acids into a growing template copy, e.g., during DNA amplification. In some embodiments, polymerases can be modified such that the active site of the polymerase is modified to reduce steric entry inhibition of the unnatural nucleic acid into the active site. In some embodiments, polymerases can be modified to provide complementarity with one or more unnatural features of the unnatural nucleic acids. Accordingly, the invention includes compositions that include a heterologous or recombinant polymerase and methods of use thereof.

Polymerases can be modified using methods pertaining to protein engineering. For example, molecular modeling can be carried out based on crystal structures to identify the locations of the polymerases where mutations can be made to modify a target activity. A residue identified as a target for replacement can be replaced with a residue selected using energy minimization modeling, homology modeling, and/or conservative amino acid substitutions, such as described in Bordo, et al. J Mol Biol 217:721-729 (1991) and Hayes, et al. Proc Natl Acad Sci, USA 99:15926-15931 (2002).

Any of a variety of polymerases can be used in a method or composition set forth herein including, for example, protein-based enzymes isolated from biological systems and functional variants thereof. Reference to a particular polymerase, such as those exemplified below, will be understood to include functional variants thereof unless indicated otherwise. In some embodiments, a polymerase is a wild type polymerase. In some embodiments, a polymerase is a modified, or mutant, polymerase.

Polymerases, with features for improving entry of unnatural nucleic acids into active site regions and for coordinating with unnatural nucleotides in the active site region, can also be used. In some embodiments, a modified polymerase has a modified nucleotide binding site.

In some embodiments, a modified polymerase has a specificity for an unnatural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the unnatural nucleic acid. In some embodiments, a modified or wild type polymerase has a specificity for an unnatural nucleic acid comprising a modified sugar that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified sugar. In some embodiments, a modified or wild type polymerase has a specificity for an unnatural nucleic acid comprising a modified base that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified base. In some embodiments, a modified or wild type polymerase has a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type polymerase toward a nucleic acid comprising a triphosphate and/or the unnatural nucleic acid without the triphosphate. For example, a modified or wild type polymerase can have a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type polymerase toward the unnatural nucleic acid with a diphosphate or monophosphate, or no phosphate, or a combination thereof.

In some embodiments, a modified or wild type polymerase has a relaxed specificity for an unnatural nucleic acid. In some embodiments, a modified or wild type polymerase has a specificity for an unnatural nucleic acid and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid. In some embodiments, a modified or wild type polymerase has a specificity for an unnatural nucleic acid comprising a modified sugar and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid. In some embodiments, a modified or wild type polymerase has a specificity for an unnatural nucleic acid comprising a modified base and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid.

Absence of exonuclease activity can be a wild type characteristic or a characteristic imparted by a variant or engineered polymerase. For example, an exo minus Klenow fragment is a mutated version of Klenow fragment that lacks 3′ to 5′ proofreading exonuclease activity.

The method of the invention may be used to expand the substrate range of any DNA polymerase which lacks an intrinsic 3 to 5′ exonuclease proofreading activity or where a 3 to 5′ exonuclease proofreading activity has been disabled, e.g. through mutation. Examples of DNA polymerases include polA, polB (see e.g. Parrel & Loeb, Nature Struc Biol 2001) polC, polD, polY, polX and reverse transcriptases (RT) but preferably are processive, high-fidelity polymerases (PCT/GB2004/004643). In some embodiments a modified or wild type polymerase substantially lacks 3′ to 5′ proofreading exonuclease activity. In some embodiments a modified or wild type polymerase substantially lacks 3′ to 5′ proofreading exonuclease activity for an unnatural nucleic acid. In some embodiments, a modified or wild type polymerase has a 3′ to 5′ proofreading exonuclease activity. In some embodiments, a modified or wild type polymerase has a 3′ to 5′ proofreading exonuclease activity for a natural nucleic acid and substantially lacks 3′ to 5′ proofreading exonuclease activity for an unnatural nucleic acid.

In some embodiments, a modified polymerase has a 3′ to 5′ proofreading exonuclease activity that is at least about 60%, 70%, 80%, 90%, 95%, 97%, 98%, 99%, 99.5%, 99.99% the proofreading exonuclease activity of the wild type polymerase. In some embodiments, a modified polymerase has a 3′ to 5′ proofreading exonuclease activity for an unnatural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for an unnatural nucleic acid and a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to the natural nucleic acid.

In a related aspect, the invention provides methods of making a modified polymerase that include structurally modeling a parental polymerase, e.g., a DNA polymerase, identifying one or more complex stability or nucleotide interaction feature affecting complex stability or nucleotide access or binding in the active site or a complementarity feature for a nucleotide analog at the active site, and mutating the parental polymerase to include or remove these features. For example, the polymerase can be mutated to improve steric access of the unnatural nucleotide to the active site or to improve charge-charge or hydrophobic interactions between the unnatural nucleotide and the polymerase. The methods also include determining whether the resulting modified polymerase displays an increased incorporation of a nucleotide or unnatural nucleotide into a growing nucleic acid copy as compared to the parental polymerase.

Polymerases can be characterized according to their rate of dissociation from nucleic acids. In some embodiments, a polymerase has a relatively low dissociation rate for one or more natural and unnatural nucleic acids. In some embodiments, a polymerase has a relatively high dissociation rate for one or more natural and unnatural nucleic acids. The dissociation rate is an activity of a polymerase that can be adjusted to tune reaction rates in methods set forth herein.

Polymerases can be characterized according to their fidelity when used with a particular natural and/or unnatural nucleic acid or collections of natural and/or unnatural nucleic acid. Fidelity generally refers to the accuracy with which a polymerase incorporates correct nucleic acids into a growing nucleic acid chain when making a copy of a nucleic acid template. DNA polymerase fidelity can be measured as the ratio of correct to incorrect natural and unnatural nucleic acid incorporations when the natural and unnatural nucleic acid are present, e.g., at equal concentrations, to compete for strand synthesis at the same site in the polymerase-strand-template nucleic acid binary complex. DNA polymerase fidelity can be calculated as the ratio of (K_{cat}/Kₘ) for the natural and unnatural nucleic acid and (kcₐₜ/Kₘ) for the incorrect natural and unnatural nucleic acid; where k_{cat} and Kₘ are Michaelis-Menten parameters in steady state enzyme kinetics (Fersht, A. R. (1985) Enzyme Structure and Mechanism, 2nd ed., p 350, W. H. Freeman & Co., New York., incorporated herein by reference). In some embodiments, a polymerase has a fidelity value of at least about 100, 1000, 10,000, 100,000, or 1×10⁶, with or without a proofreading activity.

Polymerases from native sources or variants thereof can be screened using an assay that detects incorporation of an unnatural nucleic acid having a particular structure. In one example, polymerases can be screened for the ability to incorporate an unnatural nucleic acid or UBP; e.g., d5SICSTP, dNaMTP, or d5SICSTP-dNaMTP UBP. A polymerase, e.g., a heterologous polymerase, can be used that displays a modified property for the unnatural nucleic acid as compared to the wild-type polymerase. For example, the modified property can be, e.g., Kₘ, k_{cat}, Vₘₐₓ, polymerase processivity in the presence of an unnatural nucleic acid (or of a naturally occurring nucleotide), average template read-length by the polymerase in the presence of an unnatural nucleic acid, specificity of the polymerase for an unnatural nucleic acid, rate of binding of an unnatural nucleic acid, rate of product (pyrophosphate, triphosphate, etc.) release, branching rate, or any combination thereof. In one embodiment, the modified property is a reduced Kₘ for an unnatural nucleic acid and/or an increased k_{cat}/Kₘ or Vₘₐₓ/Kₘ for an unnatural nucleic acid. Similarly, the polymerase optionally has an increased rate of binding of an unnatural nucleic acid, an increased rate of product release, and/or a decreased branching rate, as compared to a wild-type polymerase.

At the same time, a polymerase can incorporate natural nucleic acids, e.g., A, C, G, and T, into a growing nucleic acid copy. For example, a polymerase optionally displays a specific activity for a natural nucleic acid that is at least about 5% as high (e.g., 5%, 10%, 25%, 50%, 75%, 100% or higher), as a corresponding wild-type polymerase and a processivity with natural nucleic acids in the presence of a template that is at least 5% as high (e.g., 5%, 10%, 25%, 50%, 75%, 100% or higher) as the wild-type polymerase in the presence of the natural nucleic acid. Optionally, the polymerase displays a k_{cat}/Kₘ or Vₘₐₓ/Kₘ for a naturally occurring nucleotide that is at least about 5% as high (e.g., about 5%, 10%, 25%, 50%, 75% or 100% or higher) as the wild-type polymerase.

Polymerases used herein that can have the ability to incorporate an unnatural nucleic acid of a particular structure can also be produced using a directed evolution approach. A nucleic acid synthesis assay can be used to screen for polymerase variants having specificity for any of a variety of unnatural nucleic acids. For example, polymerase variants can be screened for the ability to incorporate an unnatural nucleic acid or UBP; e.g., dTPT3, dNaM analog, or dTPT3-dNaM UBP into nucleic acids. In some embodiments, such an assay is an in vitro assay, e.g., using a recombinant polymerase variant. Such directed evolution techniques can be used to screen variants of any suitable polymerase for activity toward any of the unnatural nucleic acids set forth herein.

Modified polymerases of the compositions described can optionally be a modified and/or recombinant Φ29-type DNA polymerase. Optionally, the polymerase can be a modified and/or recombinant Φ29, B103, GA-1, PZA, @15, BS32, M2Y, Nf, G1, Cp-1, PRD1, PZE, SF5, Cp-5, Cp-7, PR4, PR5, PR722, or L17 polymerase.

Nucleic acid polymerases generally useful in the invention include DNA polymerases, RNA polymerases, reverse transcriptases, and mutant or altered forms thereof. DNA polymerases and their properties are described in detail in, among other places, DNA

Replication 2^{nd} edition, Kornberg and Baker, W. H. Freeman, New York, N. Y. (1991). Known conventional DNA polymerases useful in the invention include, but are not limited to, Pyrococcus furiosus (Pfu) DNA polymerase (Lundberg et al., 1991, Gene, 108:1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (TIi) DNA polymerase (also referred to as Vent™ DNA polymerase, Cariello et al, 1991, Polynucleotides Res, 19:4193, New England Biolabs), 9°Nm™ DNA polymerase (New England Biolabs), Stoffel fragment, Thermo Sequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al, 1976, J. Bacteoriol, 127:1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from Thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al, 1981, J Biol. Chem. 256:3112), and archaeal DP1I/DP2 DNA polymerase II (Cann et al, 1998, Proc. Natl. Acad. Sci. USA 95:14250). Both mesophilic polymerases and thermophilic polymerases are contemplated. Thermophilic DNA polymerases include, but are not limited to, ThermoSequenase®, 9°Nm™, Therminator™, Taq, Tne, Tma, Pfu, TfI, Tth, TIi, Stoffel fragment, Vent™ and Deep Vent™ DNA polymerase, KOD DNA polymerase, Tgo, JDF-3, and mutants, variants and derivatives thereof. A polymerase that is a 3′ exonuclease-deficient mutant is also contemplated. Reverse transcriptases useful in the invention include, but are not limited to, reverse transcriptases from HIV, HTLV-I, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al, CRC Crit Rev Biochem. 3:289-347 (1975)). Further examples of polymerases include, but are not limited to 9°N DNA Polymerase, Taq DNA polymerase, Phusion® DNA polymerase, Pfu DNA polymerase, RB69 DNA polymerase, KOD DNA polymerase, and VentR® DNA polymerase Gardner et al. (2004) “Comparative Kinetics of Nucleotide Analog Incorporation by Vent DNA Polymerase (J. Biol. Chem., 279 (12), 11834-11842; Gardner and Jack “Determinants of nucleotide sugar recognition in an archaeon DNA polymerase” Nucleic Acids Research, 27 (12) 2545-2553.) Polymerases isolated from non-thermophilic organisms can be heat inactivatable. Examples are DNA polymerases from phage. It will be understood that polymerases from any of a variety of sources can be modified to increase or decrease their tolerance to high temperature conditions. In some embodiments, a polymerase can be thermophilic. In some embodiments, a thermophilic polymerase can be heat inactivatable. Thermophilic polymerases are typically useful for high temperature conditions or in thermocycling conditions such as those employed for polymerase chain reaction (PCR) techniques.

In some embodiments, the polymerase comprises Φ29, B103, GA-1, PZA, Φ15, BS32, M2Y, Nf, G1, Cp-1, PRD1, PZE, SF5, Cp-5, Cp-7, PR4, PR5, PR722, L17, ThermoSequenase®, 9° Nm™, Therminator™ DNA polymerase, Tne, Tma, TfI, Tth, TIi, Stoffel fragment, Vent™ and Deep Vent™ DNA polymerase, KOD DNA polymerase, Tgo, JDF-3, Pfu, Taq, T7 DNA polymerase, T7 RNA polymerase, PGB-D, UITma DNA polymerase, E. coli DNA polymerase I, E. coli DNA polymerase III, archaeal DP1I/DP2 DNA polymerase II, 9° N DNA Polymerase, Taq DNA polymerase, Phusion® DNA polymerase, Pfu DNA polymerase, SP6 RNA polymerase, RB69 DNA polymerase, Avian Myeloblastosis Virus (AMV) reverse transcriptase, Moloney Murine Leukemia Virus (MMLV) reverse transcriptase, SuperScript® II reverse transcriptase, and SuperScript® III reverse transcriptase.

In some embodiments, the polymerase is DNA polymerase 1-Klenow fragment, Vent polymerase, Phusion® DNA polymerase, KOD DNA polymerase, Taq polymerase, T7 DNA polymerase, T7 RNA polymerase, Therminator™ DNA polymerase, POLB polymerase, SP6 RNA polymerase, E. coli DNA polymerase I, E. coli DNA polymerase III, Avian Myeloblastosis Virus (AMV) reverse transcriptase, Moloney Murine Leukemia Virus (MMLV) reverse transcriptase, SuperScript® II reverse transcriptase, or SuperScript® III reverse transcriptase.

Additionally, such polymerases can be used for DNA amplification and/or sequencing applications, including real-time applications, e.g., in the context of amplification or sequencing that include incorporation of unnatural nucleic acid residues into DNA by the polymerase. In other embodiments, the unnatural nucleic acid that is incorporated can be the same as a natural residue, e.g., where a label or other moiety of the unnatural nucleic acid is removed by action of the polymerase during incorporation, or the unnatural nucleic acid can have one or more feature that distinguishes it from a natural nucleic acid.

Since at least the last common ancestor of all life on earth, genetic information has been stored in a four-letter alphabet that is propagated and retrieved by the formation of two base pairs. The central goal of synthetic biology is to create new life forms and functions, and the most general route to this goal is the creation of semi-synthetic organisms (SSOs) whose DNA harbors two additional letters that form a third, unnatural base pair (UBP). Previously, our efforts to generate such SSOs culminated in the creation of a strain of Escherichia coli that by virtue of a nucleoside triphosphate transporter from Phaeodactylum tricornutum (PINTT2), imports the requisite unnatural triphosphates from the media and then uses them to replicate a plasmid containing the UBP dNaM-dTPT3 (FIG. 1A). While the SSO stores increased information, it did not retrieve it, which requires in vivo transcription of the UBP into mRNA and tRNA, aminoacylation of the tRNA with an unnatural amino acid, and finally, efficient participation of the UBP in decoding at the ribosome. Here, we report the in vivo transcription of DNA containing dNaM and dTPT3 into mRNAs with two different unnatural codons and tRNAs with cognate unnatural anticodons, and their efficient decoding at the ribosome to direct the site-specific incorporation of natural or non-canonical amino acids (ncAAs) into superfolder green fluorescent protein (sfGFP). The results demonstrate that interactions other than hydrogen bonding can contribute to every step of information storage and retrieval. The resulting SSO both encodes and retrieves increased information and should serve as a platform for the creation of new life forms and functions.

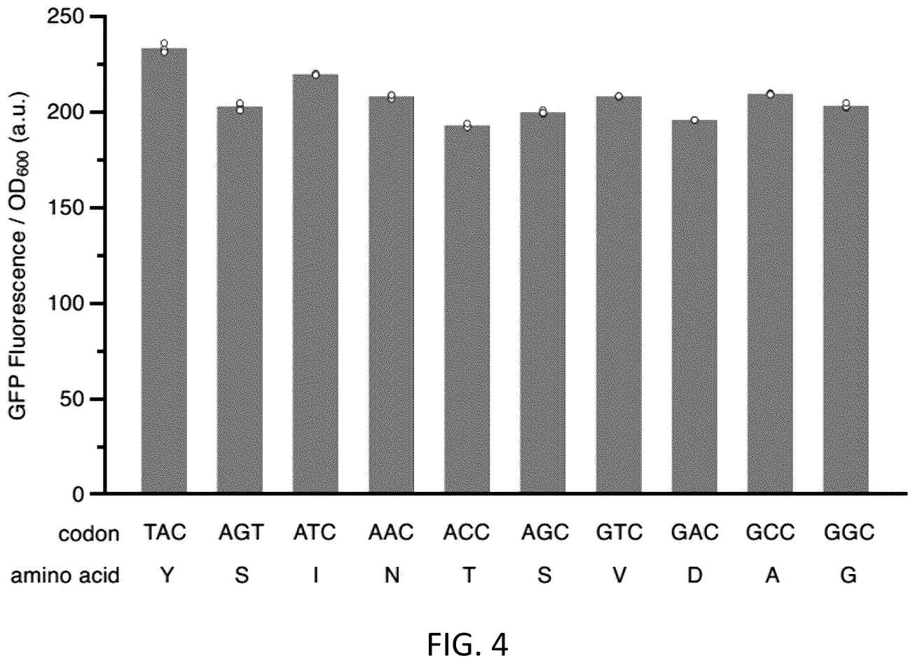

Green fluorescent protein and variants such as sfGFP have served as model systems for the study of ncAA incorporation using the amber suppression system, including at position Y151, which has been shown to tolerate a variety of natural and ncAAs (FIG. 4). To explore the decoding of unnatural codons, we first focused on the incorporation of Ser at position 151 of sfGFP, as E. coli serine aminoacyl-tRNA synthetase (SerRS) does not rely on anticodon recognition for RNA aminoacylation, thus eliminating the potential complications of inefficient charging. SSO strain YZ3 was transformed with a plasmid encoding sfGFP and an E. coli tRNA^{Ser} gene (serT), with sfGFP codon 151 (TAC) replaced by the unnatural codon AXC (sfGFP(AXC)¹⁵¹; X=NaM), and the anticodon of serT replaced by the unnatural anticodon GYT (tRNA^{Ser}(GYT); Y=TPT₃) (FIG. 1B). Transformants were grown in media supplemented with dNaMTP and dTPT3TP, then supplemented further with NaMTP and TPT3TP, as well as isopropyl-β-D-thiogalactoside (IPTG) to induce expression of T7 RNA polymerase (T7 RNAP) and tRNA^{Ser}(GYT). After a brief period of tRNA induction, anhydrotetracycline (aTc) was added to induce expression of sfGFP(AXC)¹⁵¹.

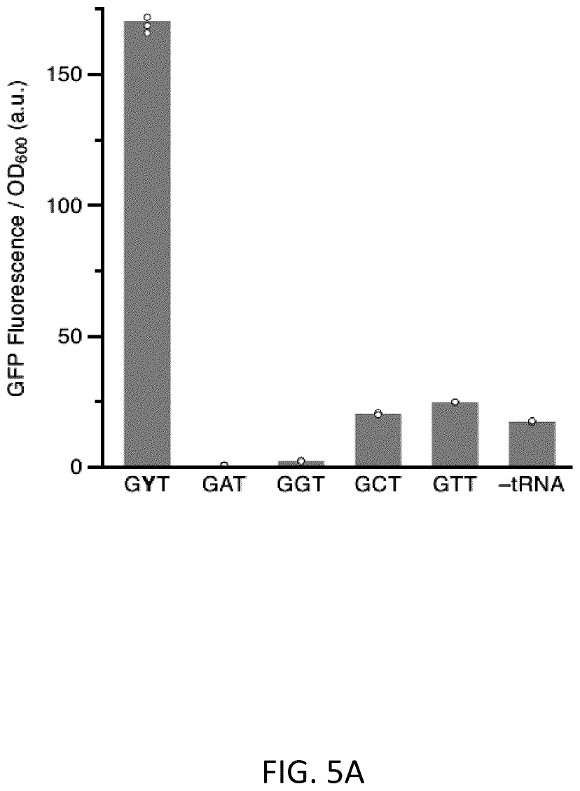

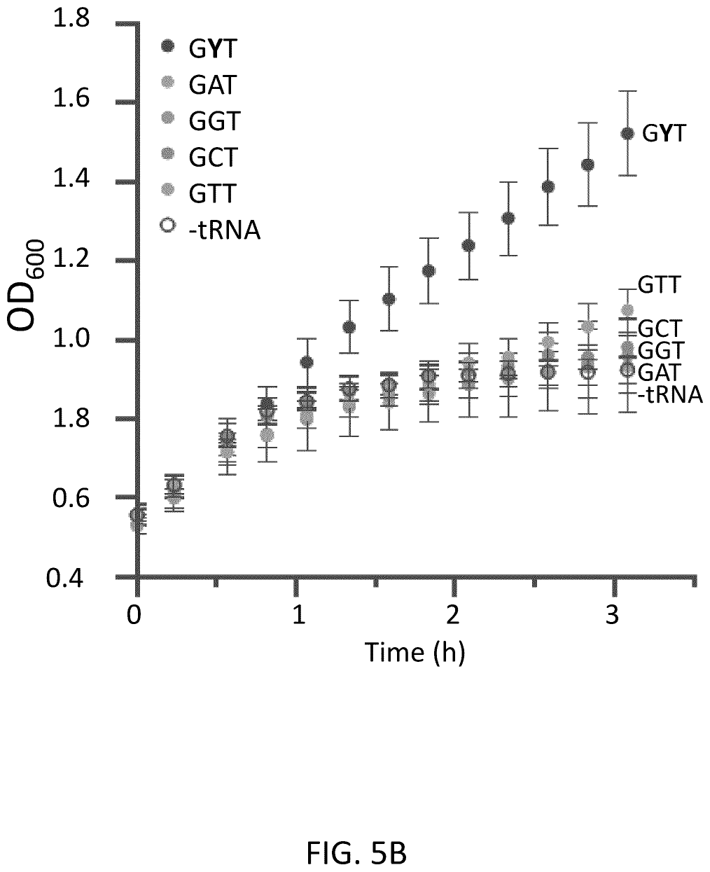

Following induction, cells transformed with a control plasmid encoding sfGFP(AXC)¹⁵¹ but lacking tRNA^{Ser}(GYT) showed dramatically reduced fluorescence compared to cells transformed with a plasmid encoding sfGFP with a natural Ser codon at position 151 (sfGFP(AGT)¹⁵¹; FIG. 1C). Moreover, cell growth began to plateau upon induction of sfGFP(AXC)¹⁵¹ (FIG. 1D), likely due to the stalling and sequestering of ribosomes. Lysates of these cells were subjected to western blotting with an anti-GFP antibody, which revealed a significant reduction in sfGFP expression and the presence of sfGFP truncated at the position of the unnatural codon (FIG. 1E). In contrast, cells transformed with the plasmid encoding both sfGFP(AXC)¹⁵¹ and tRNA^{Ser}(GYT) exhibited fluorescence that was nearly equal to that of control cells expressing sfGFP(AGT)¹⁵¹ (FIG. 1C), cell growth did not plateau upon induction of sfGFP(AXC)¹⁵¹ (FIG. 1D), and western blots of lysates from these cells revealed only full-length sfGFP protein (FIG. 1E). Furthermore, we assessed the ability of all four natural near-cognate tRNAs (TRNA^{Ser}(GNT); N=G, C, A, or T), expressed in an identical fashion, to decode the AXC codon. In each case, little fluorescence was observed and the growth defect remained (FIGS. 5A and 5B). These data demonstrate that PINTT2 is able to import both the deoxy- and ribotriphosphates of both unnatural nucleotides, that T7 RNA polymerase is able to transcribe mRNA and tRNA containing the unnatural nucleotides in vivo, and that the ribosome only efficiently decodes the unnatural codon with an unnatural anticodon.

To assess the fidelity of decoding, we analyzed protein purified from cells expressing both sfGFP(AXC)¹⁵¹ and tRNA^{Ser}(GYT) via LC/MS-MS and relative quantitation via peak intensities, which revealed a 98.5±0.7% (95% CI, n=4) incorporation of Ser at position 151, with Ile/Leu being the predominant contaminant (FIG. 1F, Table 4). Given that the retention of the UBP in the sfGFP(AXC)¹⁵¹ gene was 98±2% (95% CI, n=4) (Table 5) and that X→T is typically the major mutation during replication (which for AXC would result in the Ile codon ATC), we attribute the majority of the protein not containing Ser at position 151 to loss of the UBP during replication and conclude that the fidelity of translation with the unnatural codon is high.

TABLE 4

| Sample | S | Y | PrK | I/L | N | V | K | G | C | M |
|---|---|---|---|---|---|---|---|---|---|---|
| | Relative MS1 ion intensities (%) | | | | | | | | | |
| sfGFP(AGT)¹⁵¹ | 99.80 | 0.03 | 0.06 | 0.00 | 0.04 | 0.03 | 0.00 | 0.02 | 0.02 | 0.00 |
| sfGFP(AXC)¹⁵¹/ | 98.47 | 0.04 | 0.04 | 1.23 | 0.14 | 0.02 | 0.00 | 0.05 | 0.01 | 0.00 |
| tRNA^{Ser}(GYT) | | | | | | | | | | |
| sfGFP(TAC)¹⁵¹ | 0.11 | 99.71 | 0.06 | 0.00 | 0.05 | 0.02 | 0.00 | 0.02 | 0.02 | 0.01 |
| sfGFP(TAG)¹⁵¹/ | 0.06 | 0.04 | 99.53 | 0.00 | 0.04 | 0.01 | 0.29 | 0.01 | 0.01 | 0.00 |
| tRNA^{Pyl}(CTA) | | | | | | | | | | |
| sfGFP(AXC)¹⁵¹/ | 0.25 | 0.03 | 96.16 | 2.06 | 1.06 | 0.02 | 0.37 | 0.03 | 0.01 | 0.00 |
| tRNA^{Pyl}(GYT) | | | | | | | | | | |
| sfGFP(GXC)¹⁵¹/ | 0.06 | 0.04 | 97.50 | 0.00 | 0.01 | 1.26 | 0.74 | 0.37 | 0.01 | 0.00 |
| tRNA^{Pyl}(GYC) | | | | | | | | | | |
| | 95% CI (%) | | | | | | | | | |
| sfGFP(AGT)¹⁵¹ | 0.31 | 0.04 | 0.09 | 0.00 | 0.06 | 0.05 | 0.01 | 0.03 | 0.03 | 0.00 |
| sfGFP(AXC)¹⁵¹/ | 0.73 | 0.04 | 0.03 | 0.64 | 0.04 | 0.01 | 0.00 | 0.04 | 0.01 | 0.00 |
| tRNA^{Ser}(GYT) | | | | | | | | | | |
| sfGFP(TAC)¹⁵¹ | 0.06 | 0.11 | 0.05 | 0.00 | 0.03 | 0.02 | 0.00 | 0.01 | 0.02 | 0.00 |
| sfGFP(TAG)¹⁵¹/ | 0.03 | 0.02 | 0.11 | 0.00 | 0.02 | 0.02 | 0.03 | 0.01 | 0.01 | 0.00 |
| tRNA^{Pyl}(CTA) | | | | | | | | | | |
| sfGFP(AXC)¹⁵¹/ | 0.13 | 0.02 | 0.25 | 0.06 | 0.03 | 0.01 | 0.06 | 0.01 | 0.02 | 0.01 |
| tRNA^{Pyl}(GYT) | | | | | | | | | | |
| sfGFP(GXC)¹⁵¹/ | 0.05 | 0.04 | 0.70 | 0.00 | 0.01 | 0.24 | 0.28 | 0.22 | 0.01 | 0.00 |
| tRNA^{Pyl}(GYC) | | | | | | | | | | |

TABLE 5

| | | | | | % UBP | | |
|---|---|---|---|---|---|---|---|
| | | | | | Retention | Anti. | % UBP Retention |
| aaRS | tRNA | NaMTP | TPT3TP | Codon | sfGFP | codon | in tRNA gene |
| SerRS | − | + | + | AXC | 98 ± 0 | − | n/a |
| SerRS§ | Ser | + | + | AXC | 98 ± 2 | GYT | 89 ± 2 |
| SerRS | Ser | + | + | AXC | 94 ± 8 | GAT | n/a |
| SerRS | Ser | + | + | AXC | 94 ± 2 | GGT | n/a |
| SerRS | Ser | + | + | AXC | 95 ± 0 | GCT | n/a |
| SerRS | Ser | + | + | AXC | 95 ± 1 | GTT | n/a |
| − | Pyl | + | + | AXC | 97 ± 1 | GYT | 89 ± 2 |
| PylRS | − | + | + | AXC | 97 ± 1 | − | n/a |
| PylRS | Pyl | + | + | TAC | n/a | GYT | 92 ± 3 |
| PylRS§ | Pyl | + | + | AXC | 96 ± 1 | GYT | 90 ± 2 |
| PylRS* | Pyl | + | + | AXC | 98 ± 0 | GYT | 95 ± 2 |
| PylRS* | Pyl | + | − | AXC | 98 ± 1 | GYT | 96 ± 1 |
| PylRS* | Pyl | − | + | AXC | 98 ± 1 | GYT | 95 ± 1 |
| PylRS* | Pyl | − | − | AXC | 97 ± 1 | GYT | 94 ± 4 |
| − | Pyl | + | + | GXC | 98 ± 1 | GYC | 96 ± 3 |
| PylRS | − | + | + | GXC | 97 ± 3 | − | n/a |
| PylRS | Pyl | + | + | TAC | n/a | GYC | 96 ± 1 |
| PylRS§ | Pyl | + | + | GXC | 97 ± 1 | GYC | 95 ± C |
| PylRS* | Pyl | + | + | GXC | 96 ± 3 | GYC | 97 ± 1 |
| PylRS* | Pyl | + | − | GXC | 96 ± 2 | GYC | 97 ± 1 |
| PylRS* | Pyl | − | + | GXC | 97 ± 2 | GYC | 97 ± 0 |
| PylRS* | Pyl | − | − | GXC | 96 ± 1 | GYC | 97 ± 1 |
| pAzFRS RS§ | pAzF | + | + | AXC | 98 ± 0 | GYT | 90 ± 1 |
| pAzFRS RS | pAzF | + | + | TAC | n/a | GYT | 91 ± 1 |

*Corresponds to the cultures analyzed in FIGS. 7A-7D.

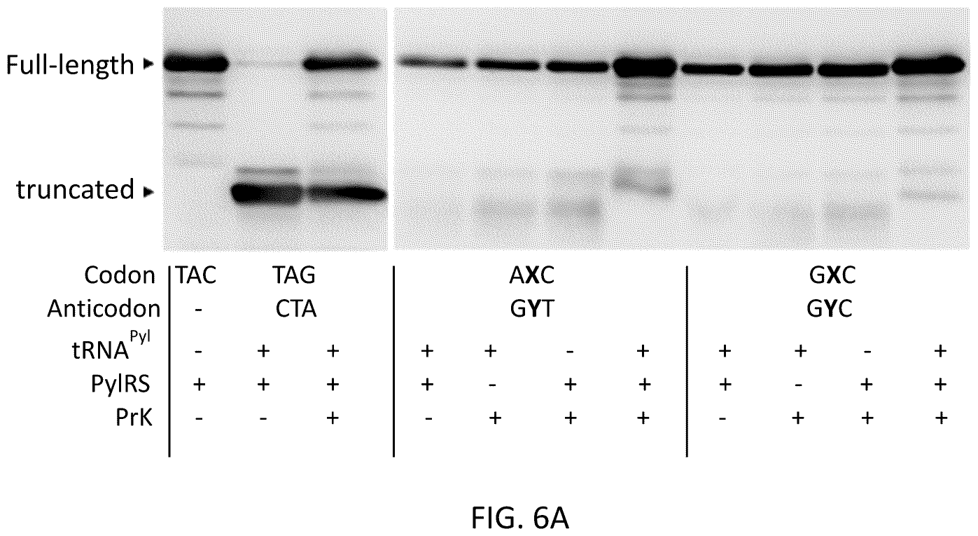

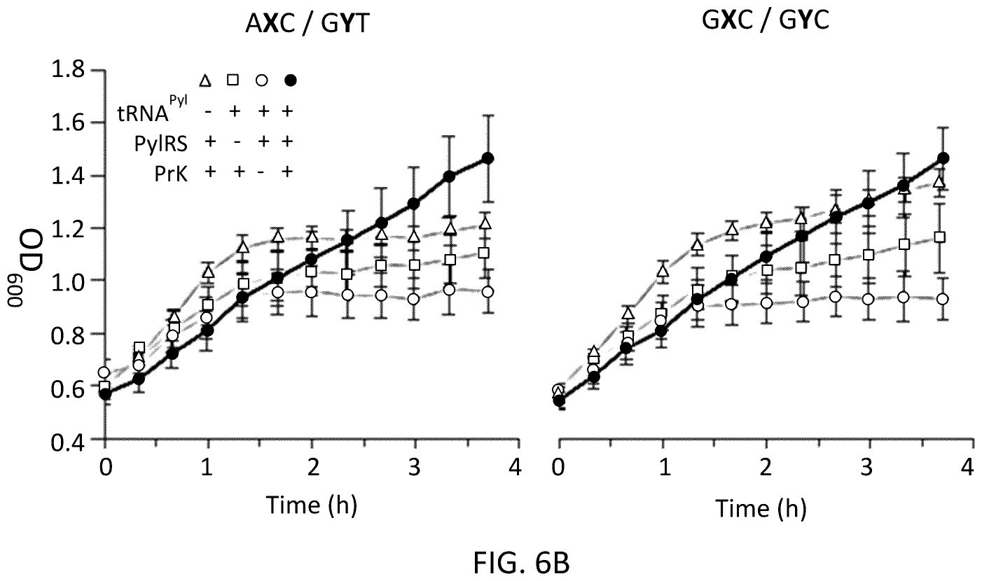

To demonstrate the encoding of ncAAs with UBPs, we constructed plasmids analogous to those used above, but with the tRNA^{Ser} gene replaced with the Methanosarcina mazei tRNA^{Pyl}(GYT) gene. tRNA^{Pyl} can be selectively charged by the Methanosarcina barkeri pyrrolysine aminoacyl tRNA synthetase (PylRS) with the ncAA N⁶-(2-propynyloxy)carbonyl]-L-lysine (PrK). In addition to the codon AXC, we also analyzed the codon GXC and the corresponding tRNA^{Pyl}(GYC). The SSO, carrying a separate plasmid encoding an IPTG-inducible PylRS, was transformed with the required plasmids and grown with or without added PrK. In control experiments with cells expressing either sfGFP(AXC)¹⁵¹ or sfGFP(GXC)¹⁵¹ in the absence of either PylRS, the cognate unnatural tRNA^{Pyl}, or PrK, we observed only low cellular fluorescence (FIG. 2A), truncation of sfGFP (FIGS. 6A and 6B), and a plateau in cell growth (FIG. 6B). In contrast, for either unnatural mRNA with its cognate unnatural tRNA, when PylRS was present and PrK was added, we observed high fluorescence (64% and 69% of sfGFP(TAC)¹⁵¹ for AXC and GXC, respectively) (FIGS. 2A and 2B), robust production of full-length sfGFP (FIG. 6A), and normal growth (FIG. 6B).

To verify the incorporation of PrK, sfGFP was affinity purified from cell lysates using a C-terminal Strep-tag II and subjected to copper-catalyzed click chemistry to attach a carboxytetramethylrhodamine (TAMRA) dye (TAMRA-PEG₄-N₃), which was found to shift the electrophoretic mobility of sfGFP during SDS-PAGE, thus allowing us to assess the fidelity of PrK incorporation by western blotting (FIG. 2C). We observed strong TAMRA signal and that virtually all of the sfGFP was shifted when purified from cells expressing sfGFP(AXC)¹⁵¹ and tRNA^{Pyl}(GYT) or sfGFP(GXC)¹⁵¹ and tRNA^{Pyl}(GYC), and which had been cultured in media supplemented with PrK (FIG. 2C). In contrast, little to no TAMRA signal or shifted sfGFP was observed when NaMTP, TPT3TP, or both were absent (FIGS. 7A and 7B). Finally, no TAMRA signal or shifted sfGFP was observed in protein purified from cells expressing sfGFP (TAC) 151 with either unnatural tRNA (FIG. 2C). This data demonstrates that PrK is specifically incorporated into sfGFP via decoding of the unnatural codons by tRNAs with an unnatural anticodon.

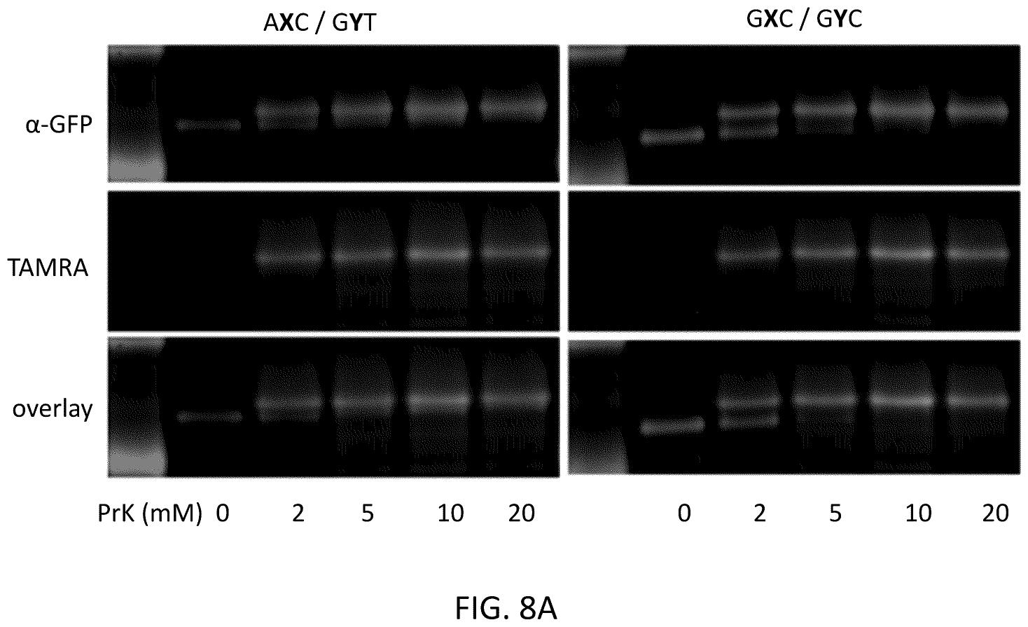

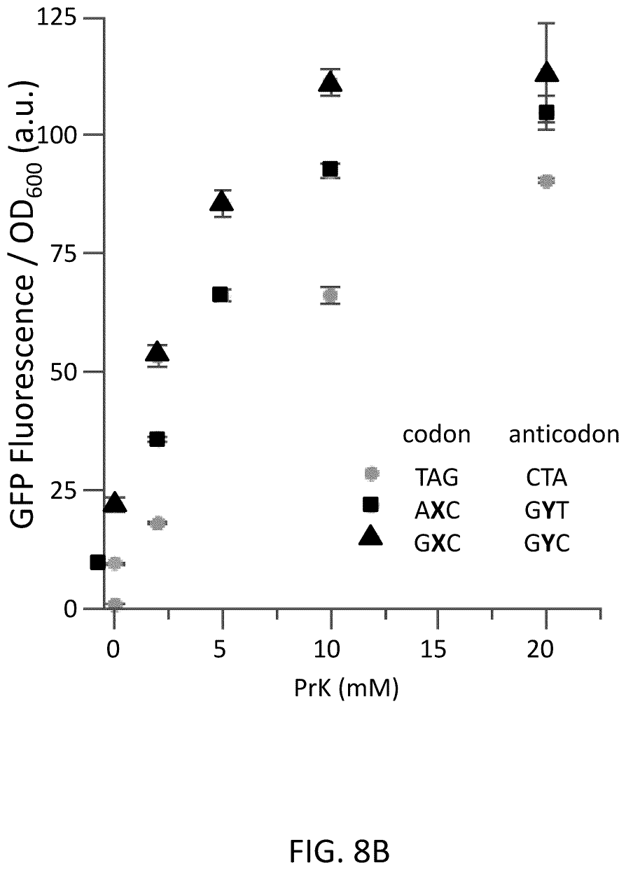

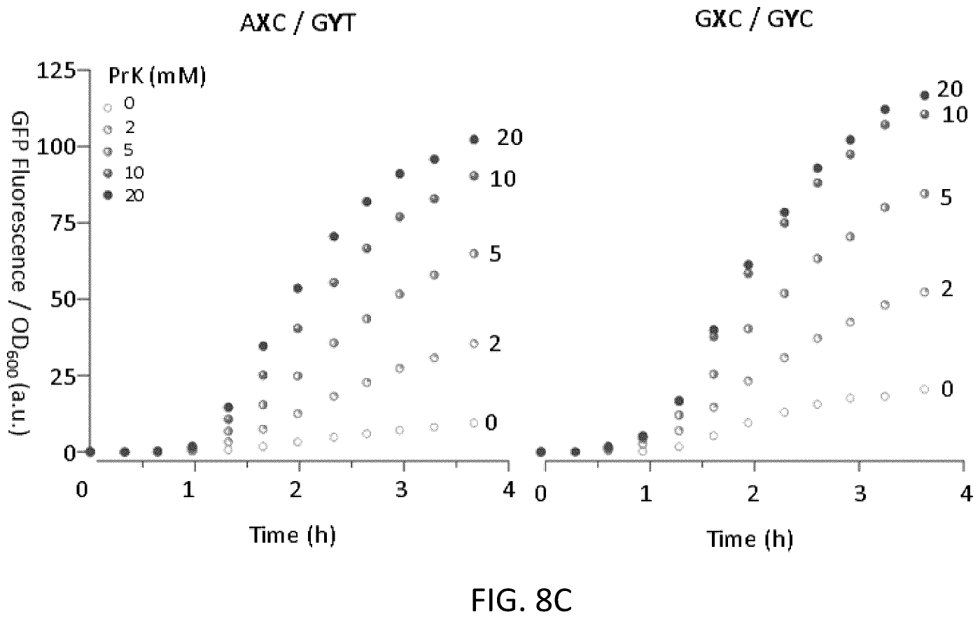

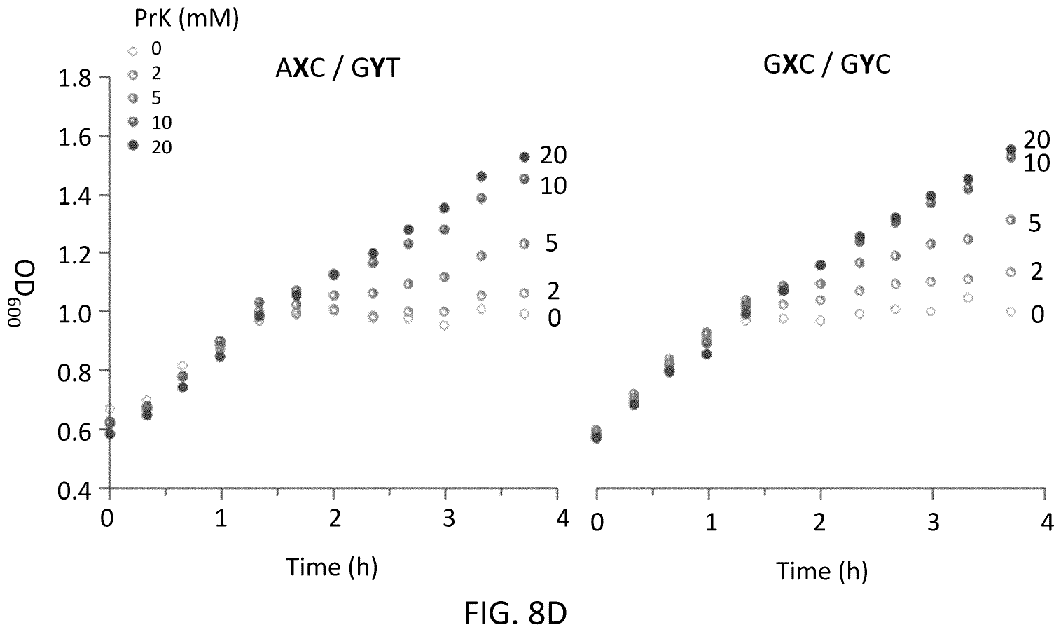

With optimal PrK concentrations (FIGS. 8A-8D), we purified 54±4 and 55±6 μg/mL of sfGFP (s.d., n=4, ˜40% of the sfGFP (TAC)¹⁵¹ control (Table 6) for the AXC and GXC codons, respectively. Moreover, based on mass spectrometry analysis, the purity of sfGFP with PrK was 96.2±0.3% (95% CI, n=4) for the AXC codon and 97.5±0.7% (95% CI, n=4) for the GXC codon (FIG. 2D). Although the yield of sfGFP protein purified was slightly lower than with amber suppression (87±6 μg/mL, s.d., n=4 (Table 6)), due to a moderate reduction in growth with addition of the unnatural ribotriphosphates (FIGS. 7C and 7D), decoding of both unnatural codons resulted in higher fluorescence than amber suppression when normalized to cell density (FIGS. 2A and 2B), implying that decoding with the unnatural codons is more efficient than amber suppression.

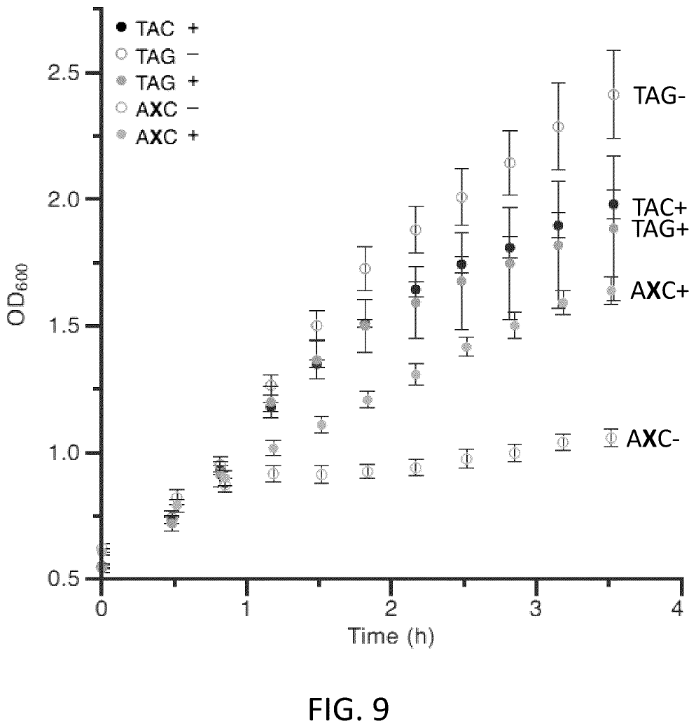

To explore the encoding of other ncAAs with UBPs, we examined the encoding of p-azido-phenylalanine (pAzF) with the AXC codon and an evolved Methanococcus jannaschii TyrRS/tRNA^{Tyr} pair (pAzFRS/tRNA^{pAzF}). With induction of the synthetase and the addition of pAzF to the growth media, we observed robust fluorescence equivalent to that of cells expressing natural sfGFP(TAC)¹⁵¹ and normal growth with sfGFP(AXC)¹⁵¹ and tRNA^{pAzF}(GYT) (FIG. 3A, FIG. 9). Full-length sfGFP was purified (86±6 μg/mL, s.d., n=4; 68% of the sfGFP (TAC) 151 control, Table 6) and subjected to copper-free click chemistry using a dibenzocyclooctyl (DBCO) group to attach TAMRA (TAMRA-PEG₄-DBCO). We observed robust TAMRA conjugation to sfGFP isolated from cells expressing sfGFP(AXC)¹⁵¹ and tRNA^{pAzF}(GYT) and cultured in the presence of pAzF (FIG. 3B). Although we were unable to accurately assess the fidelity of pAzF incorporation due to decomposition of the azido moiety, ˜93% of the sfGFP protein was shifted, which compares favorably to the ˜95% shifted sfGFP produced via amber suppression (FIG. 3B).

TABLE 6

| | | | Relative | Total | Relative |
|---|---|---|---|---|---|
| | | Yield | to control | Fluor | to control |
| Sample | aaRS | (μg/mL) | (%) | (a.u.) | (%) |
| sfGFP(AGT)¹⁵¹ | SerRS | 100 ± 8 | 100 | 269 | 100 |
| sfGFP(AXC)¹⁵¹/tRNA^{Ser}(GYT) | (endogenous) | 97 ± 9 | 96 | 259 | 96 |
| sfGFP(TAC)¹⁵¹ | PylRS | 135 ± 17 | 100 | 400 | 100 |
| sfGFP(TAG)¹⁵¹/tRNA^{Pyl}(CTA) | | 87 ± 6 | 65 | 242 | 60 |
| sfGFP(AXC)¹⁵¹/tRNA^{Pyl}(GYT) | | 54 ± 4 | 40 | 153 | 38 |
| sfGFP(GXC)¹⁵¹/tRNA^{Pyl}(GYC) | | 55 ± 6 | 41 | 166 | 41 |
| sfGFP(TAC)¹⁵¹ | pAzFRS | 127 ± 15 | 100 | 405 | 100 |
| sfGFP(TAG)¹⁵¹/tRNA^{pAzF}(CTA) | | 75 ± 9 | 59 | 287 | 71 |
| sfGFP(AXC)¹⁵¹/tRNA^{pAzF}(GYT) | | 86 ± 6 | 68 | 333 | 82 |

Since at least the last common ancestor of all life on earth, proteins have been produced via the decoding of codons written solely with the four-nucleotide genetic alphabet. We have now demonstrated the decoding of two new codons, written with an expanded genetic alphabet, and used the new codons to site-specifically incorporate ncAAs into proteins. We find that for every step of information storage and retrieval, hydrogen bonds, so obviously central to the natural base pairs, may at least in part be replaced with complementary packing and hydrophobic forces. Despite their novel mechanism of decoding, the unnatural codons can be decoded as efficiently as their fully natural counterparts. While we have only examined the decoding of two unnatural codons, the UBP is unlikely to be limited to these, and when combined with a recently reported Cas9 editing system that reinforces UBP retention, it will likely make available more codons than can ever be used. Thus, the reported SSO may be just the first of a new form of semi-synthetic life that is able to access a broad range of forms and functions not available to natural organisms.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A cell comprising:
a. a nucleoside triphosphate transporter from Phaeodactylum tricornutum;
b. a tRNA from Methanosarcina mazei or Methanococcus jannaschii comprising an anticodon, the anticodon comprising an unnatural nucleobase selected from 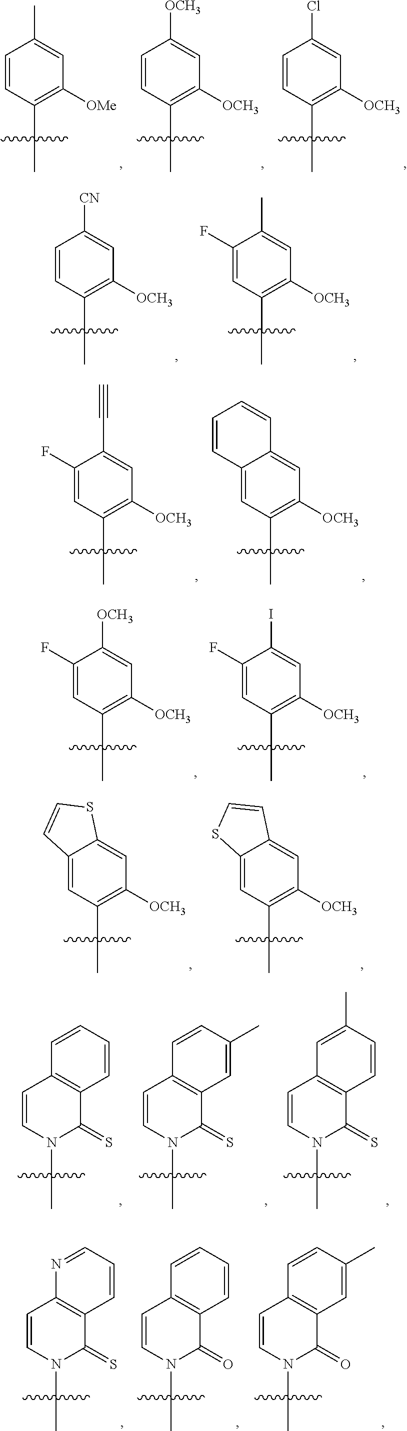 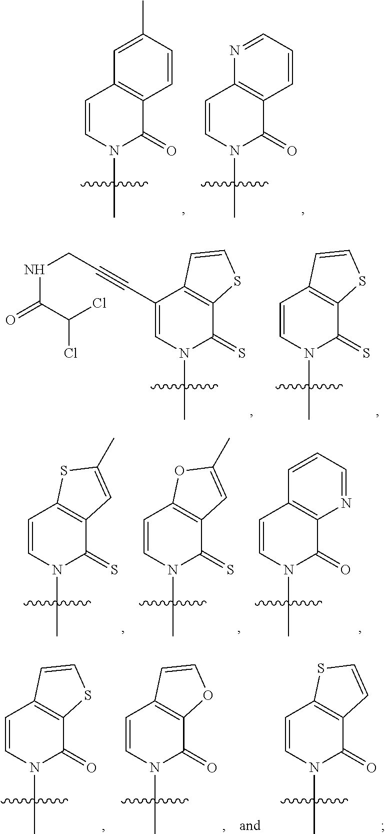
c. an aminoacyl tRNA synthetase; and
d. an mRNA comprising a codon, the codon comprising an unnatural nucleobase selected from

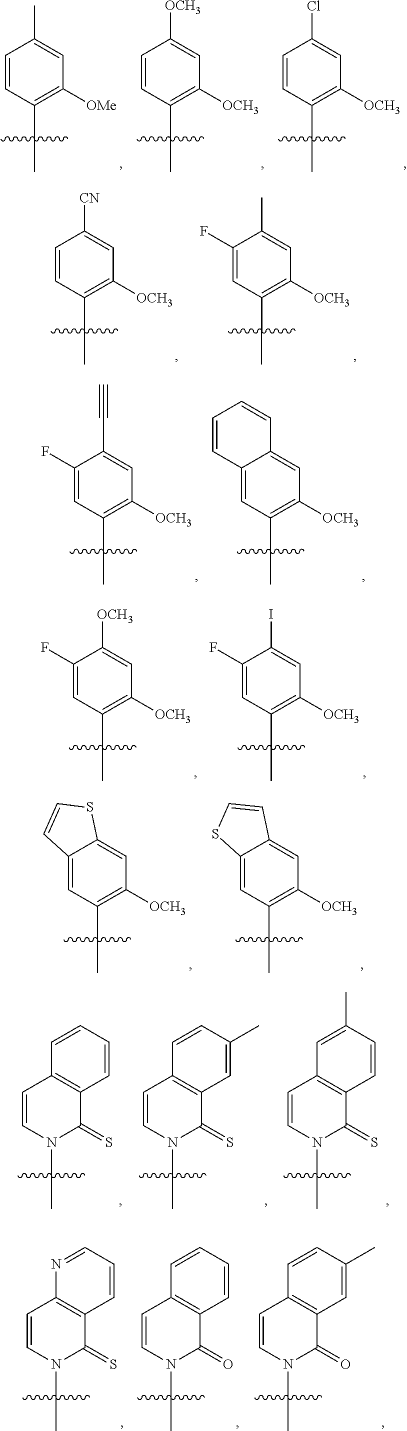

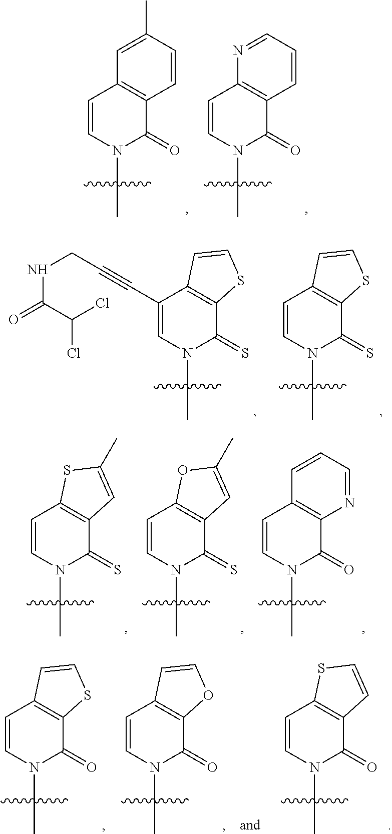

2. The cell of claim 1, wherein the cell further comprises an oligonucleotide encoding the tRNA.

3. The cell of claim 1, wherein the cell further comprises an oligonucleotide encoding the aminoacyl tRNA synthetase.

4. The cell of claim 1, wherein the cell further comprises an oligonucleotide encoding the mRNA.

5. The cell of claim 1, wherein the cell further comprises an oligonucleotide that encodes the tRNA and the mRNA.

6. The cell of claim 1, wherein the cell further comprises an oligonucleotide that encodes the tRNA, the mRNA, and the aminoacyl tRNA synthetase.

7. The cell of claim 1, wherein the unnatural nucleobase in the anticodon is 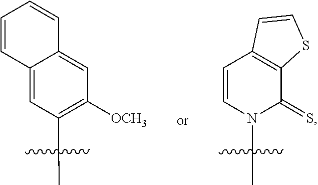 [SMILES: *c1cc2ccccc2cc1OC ; *n1ccc2ccsc2c1=S]
and the unnatural nucleobase in the codon is

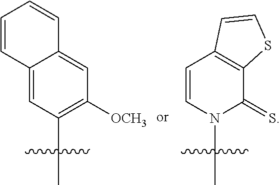 [SMILES: *c1cc2ccccc2cc1OC ; "or" ; *n1ccc2ccsc2c1=S]

8. The cell of claim 7, wherein the unnatural nucleobase in the codon is 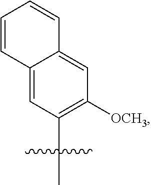 [SMILES: *c1cc2ccccc2cc1OC]
and the unnatural nucleobase in the anticodon is

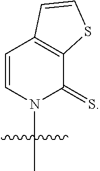 [SMILES: *n1ccc2ccsc2c1=S]

9. The cell of claim 1, wherein the anticodon comprises the sequence GYT or GYC, wherein Y comprises the unnatural nucleobase.

10. The cell of claim 9, wherein the unnatural nucleobase of Y is

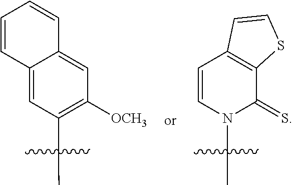 [SMILES: *c1cc2ccccc2cc1OC ; *n1ccc2ccsc2c1=S]

11. The cell of claim 10, wherein the unnatural nucleobase of Y is

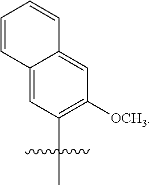 [SMILES: *c1cc2ccccc2cc1OC]

12. The cell of claim 10, wherein the unnatural nucleobase of Y is

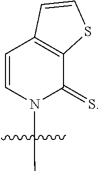 [SMILES: *n1ccc2ccsc2c1=S]

13. The cell of claim 1, wherein the codon comprises the sequence AXC or GXC, wherein X comprises the unnatural nucleobase.

14. The cell of claim 13, wherein the codon comprises the sequence AXC.

15. The cell of claim 14, wherein the unnatural nucleobase of X is

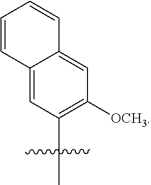 [SMILES: *c1cc2ccccc2cc1OC]

16. The cell of claim 14, wherein the unnatural nucleobase of X is

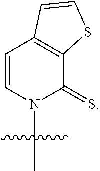 [SMILES: *n1ccc2ccsc2c1=S]

17. The cell of claim 13, wherein the codon comprises the sequence GXC.

18. The cell of claim 17, wherein the unnatural nucleobase of X is

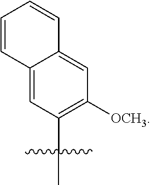 [SMILES: *c1cc2ccccc2cc1OC]

19. The cell of claim 17, wherein the unnatural nucleobase of X is

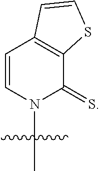 [SMILES: *n1ccc2ccsc2c1=S]

20. The cell of claim 1, wherein the aminoacyl tRNA synthetase is from Methanosarcina barkeri.

21. The cell of claim 1, wherein the aminoacyl tRNA synthetase is from Methanococcus jannaschii.

22. The cell of claim 1, wherein the aminoacyl tRNA synthetase is a pyrrolysyl tRNA synthetase.

23. The cell of claim 1, wherein the aminoacyl tRNA synthetase is a tyrosyl tRNA synthetase.

24. The cell of claim 1, wherein the cell is a microorganism or bacterium.

25. The cell of claim 1, wherein the cell is an E. coli cell.

26. A method of producing a protein comprising an unnatural amino acid in a cell, the cell comprising:
(i) a nucleoside triphosphate transporter from Phaeodactylum tricornutum;
(ii) a tRNA from Methanosarcina mazei or Methanococcus jannaschii comprising an anticodon, the anticodon comprising an unnatural nucleobase selected from 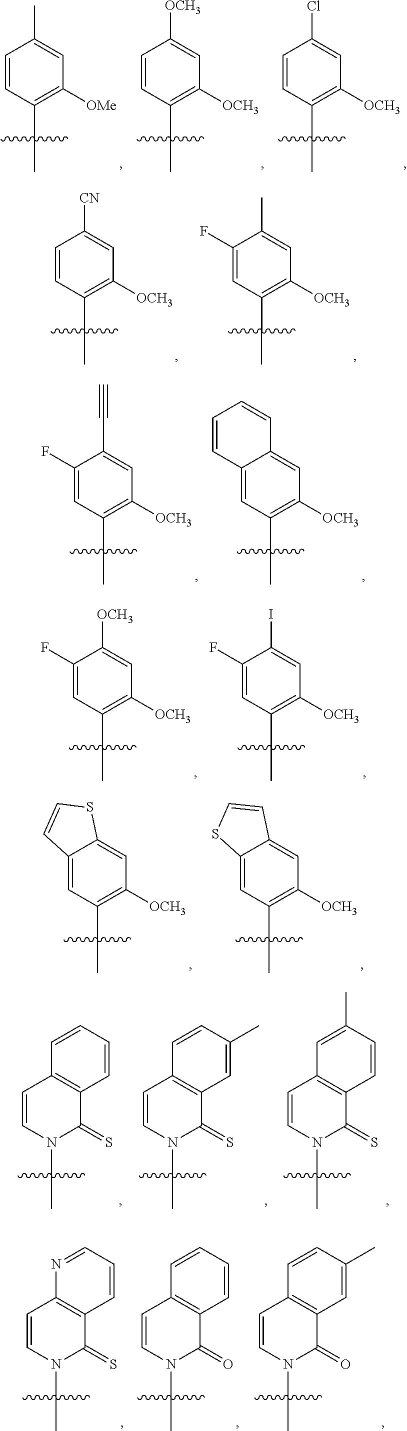 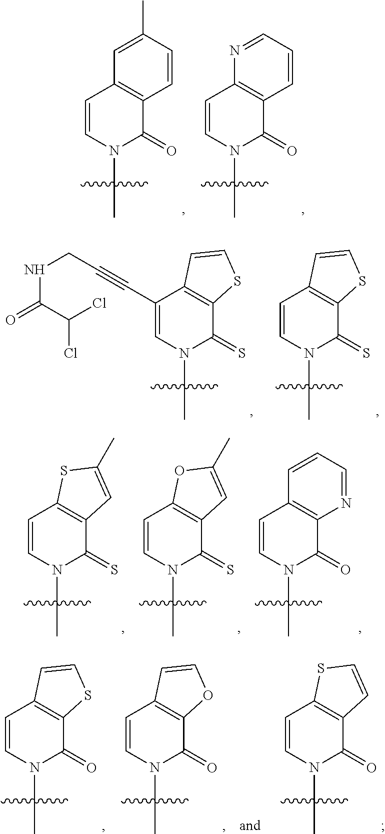
(iii) an aminoacyl tRNA synthetase;
(iv) an mRNA comprising a codon, the codon comprising an unnatural nucleobase selected from 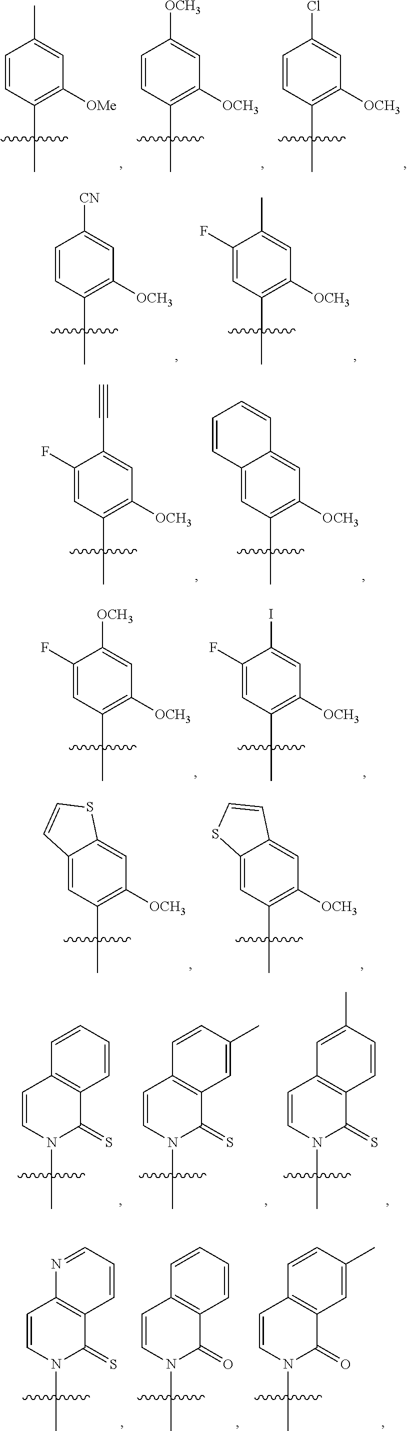 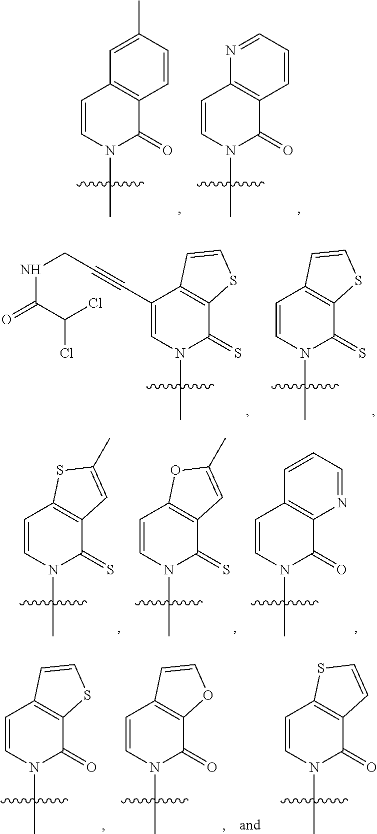
and
(v) the unnatural amino acid;
the method comprising synthesizing the protein in the cell by translating the protein from the mRNA.

27. The method of claim 26, further comprising transporting an unnatural nucleotide into the cell via the nucleoside triphosphate transporter.

28. The method of claim 26, wherein the unnatural nucleobase in the anticodon or the codon is

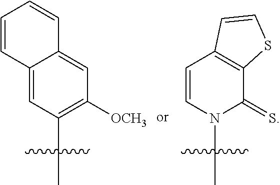 [SMILES: *c1cc2ccccc2cc1OC ; "or" ; *n1ccc2ccsc2c1=S]

29. The method of claim 26, further comprising forming an unnatural base pair between a first nucleotide and a second nucleotide, wherein the first nucleotide comprises a nucleobase having a structure 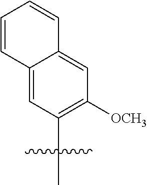 [SMILES: *c1cc2ccccc2cc1OC]
and the second nucleotide comprises a nucleobase having a structure

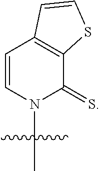 [SMILES: *n1ccc2ccsc2c1=S]

30. The method of claim 26, wherein the anticodon comprises the sequence GYT or GYC, wherein Y comprises the unnatural nucleobase.

31. The method of claim 30, wherein the unnatural nucleobase of Y is

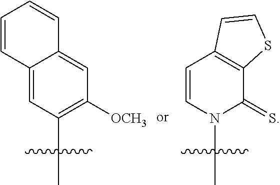 [SMILES: *c1cc2ccccc2cc1OC ; "or" ; *n1ccc2ccsc2c1=S]

32. The method of claim 26, wherein the aminoacyl tRNA synthetase is from Methanosarcina barkeri.

33. The method of claim 26, wherein the aminoacyl tRNA synthetase is from Methanococcus jannaschii.

34 The method of claim 26, wherein the cell is a microorganism or bacterium.

35. The method of claim 26, wherein the cell is E. coli.

36. A method of producing a protein comprising an unnatural amino acid in a cell, the cell comprising:
(i) a nucleoside triphosphate transporter from Phaeodactylum tricornutum;
(ii) a tRNA from Methanosarcina mazei or Methanococcus jannaschii comprising an anticodon, the anticodon comprising an unnatural nucleobase selected from 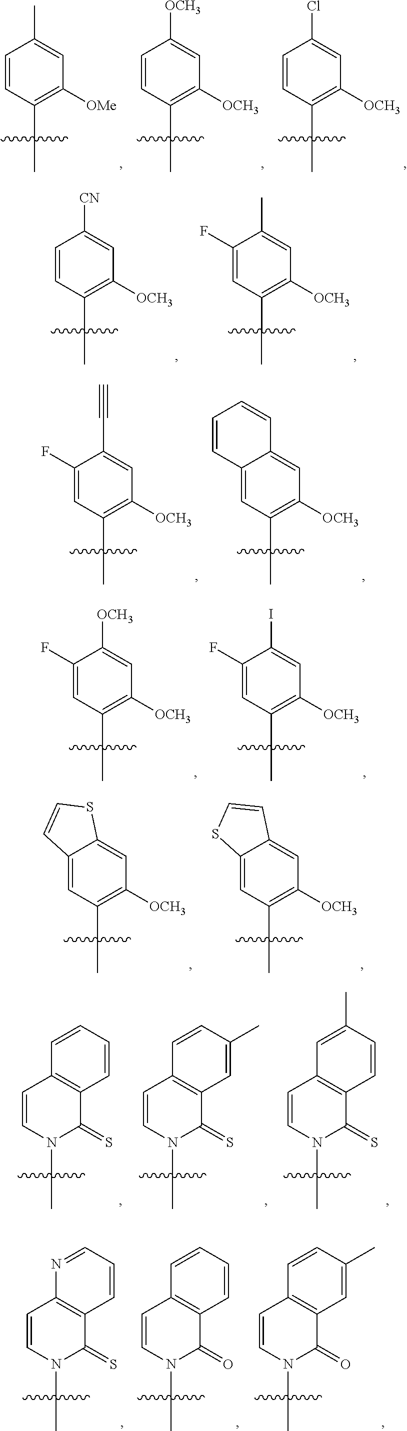 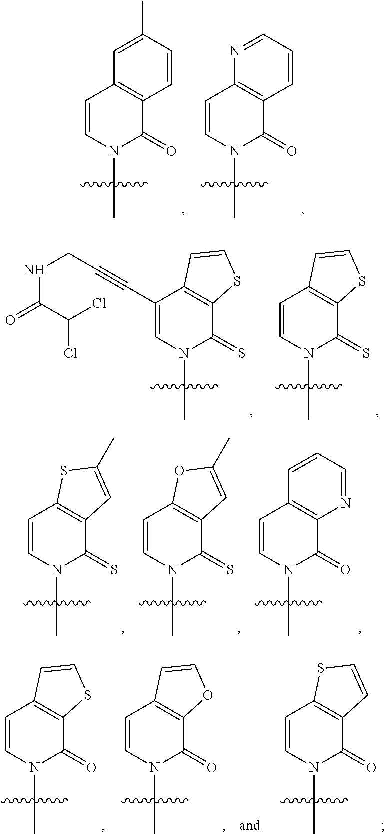
(iii) an aminoacyl tRNA synthetase;
(iv) a nucleic acid encoding an mRNA comprising a codon, the codon comprising an unnatural nucleobase selected from 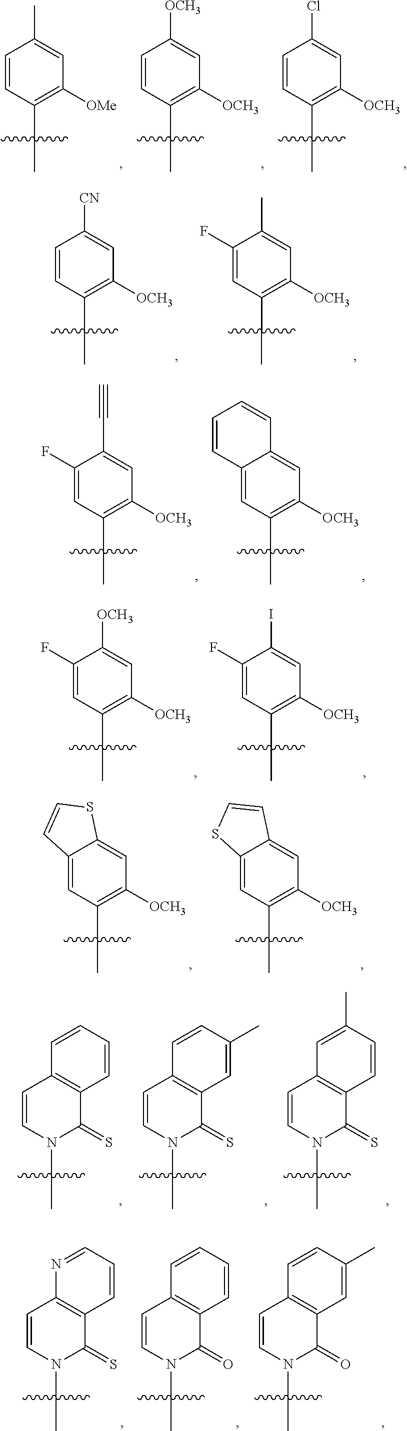 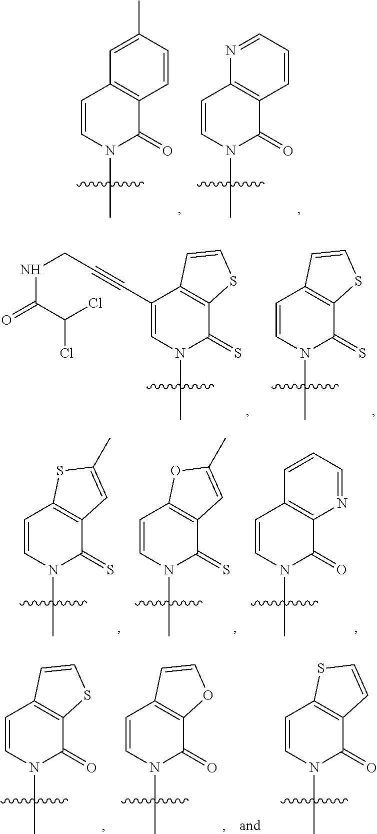
and
(v) the unnatural amino acid;
the method comprising
a) synthesizing the mRNA comprising the codon, wherein an unnatural nucleotide is incorporated during transcription; and
b) synthesizing the protein in the cell by translating the protein from the mRNA.

37. The method of claim 36, further comprising transporting the unnatural nucleotide into the cell via the nucleoside triphosphate transporter.

38. The method of claim 36, wherein the unnatural nucleobase in the anticodon or the codon is

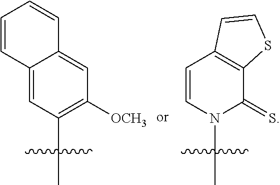 [SMILES: *c1cc2ccccc2cc1OC ; "or" ; *n1ccc2ccsc2c1=S]

39. The method of claim 36, further comprising forming an unnatural base pair with a first nucleotide and a second nucleotide, wherein the first nucleotide comprises a nucleobase having a structure 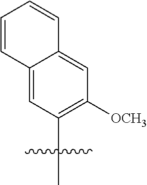 [SMILES: *c1cc2ccccc2cc1OC]
and the second nucleotide comprises a nucleobase having a structure

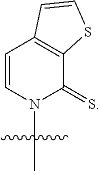 [SMILES: *n1ccc2ccsc2c1=S]

40. The method of claim 36, wherein the anticodon comprises the sequence GYT or GYC, wherein Y comprises the unnatural nucleobase.

41. The method of claim 36, wherein the unnatural nucleobase of Y is

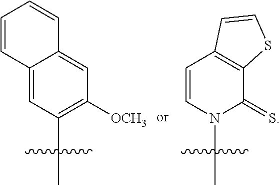 [SMILES: *c1cc2ccccc2cc1OC ; "or" ; *n1ccc2ccsc2c1=S]

42. The method of claim 36, wherein the cell is a microorganism or bacterium.

43. The method of claim 36, wherein the cell is E. coli.

44. A method of producing a protein comprising an unnatural amino acid in a cell, the cell comprising:
(i) a nucleoside triphosphate transporter from Phaeodactylum tricornutum;
(ii) a nucleic acid encoding a tRNA from Methanosarcina mazei or Methanococcus jannaschii comprising an anticodon, the anticodon comprising an unnatural nucleobase selected from 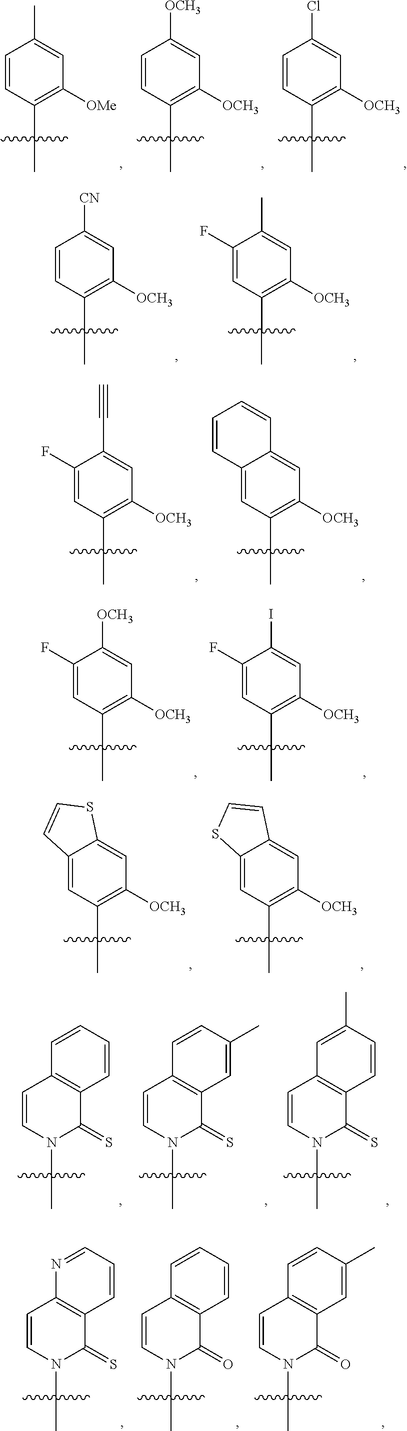 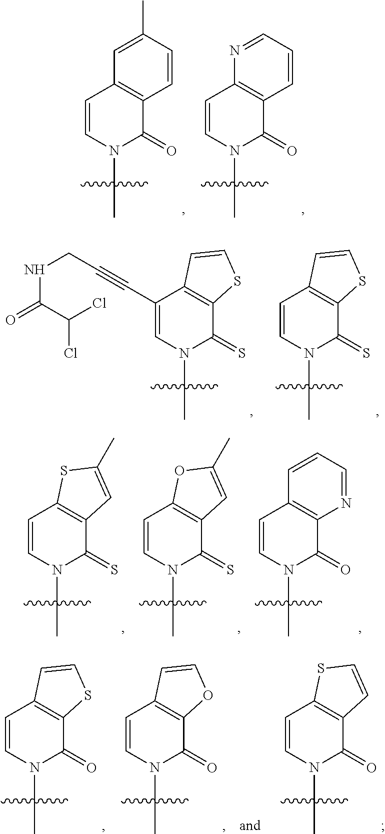
(iii) an aminoacyl tRNA synthetase;
(iv) a nucleic acid encoding an mRNA comprising a codon, the codon comprising an unnatural nucleobase selected from 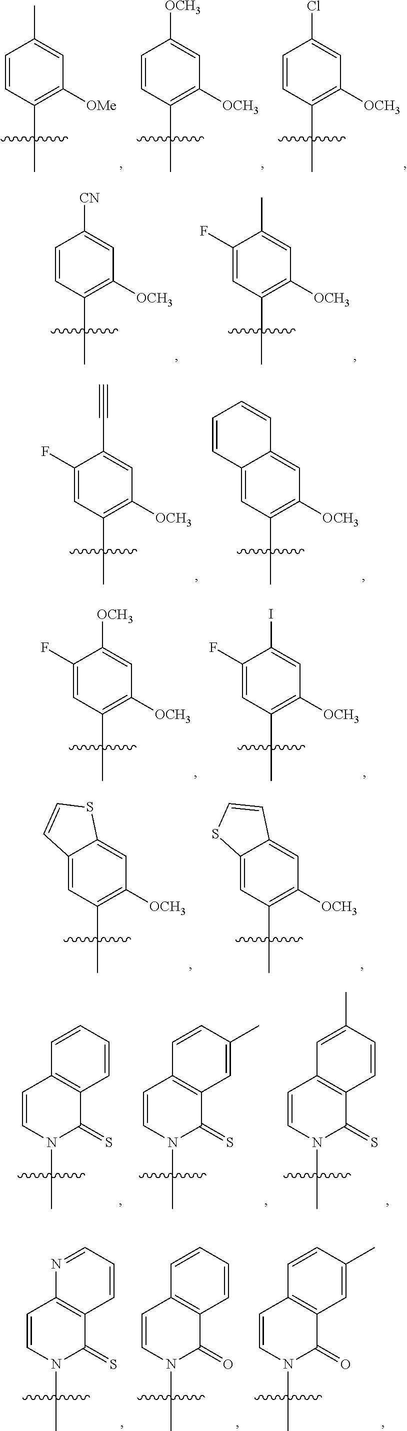 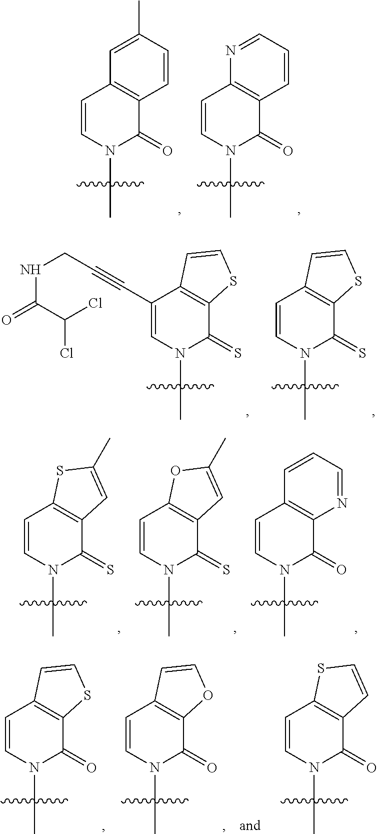
and
(v) the unnatural amino acid;
the method comprising
a) synthesizing the tRNA comprising the anticodon and the mRNA comprising the codon, wherein a first unnatural nucleotide is incorporated into the tRNA during transcription and a second unnatural nucleotide is incorporated into the mRNA during transcription; and
b) synthesizing the protein in the cell by translating the protein from the mRNA.

45. The method of claim 44, further comprising transporting an unnatural nucleotide into the cell via the nucleoside triphosphate transporter.

46. The method of claim 44, further comprising expressing the aminoacyl tRNA synthetase from a plasmid in the cell.

47. The method of claim 44, wherein the unnatural nucleobase in the anticodon or the codon is

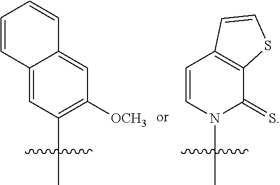 [SMILES: *c1cc2ccccc2cc1OC ; "or" ; *n1ccc2ccsc2c1=S]

48. The method of claim 44, further comprising forming an unnatural base pair between a nucleotide comprising a nucleobase having a structure 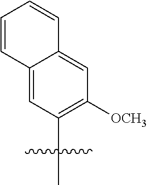 [SMILES: *c1cc2ccccc2cc1OC]
and a nucleotide comprising a nucleobase having a structure

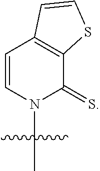 [SMILES: *n1ccc2ccsc2c1=S]

49. The method of claim 44, wherein the anticodon comprises from the sequence GYT or GYC, wherein Y comprises the unnatural nucleobase.

50. The method of claim 49, wherein Y is

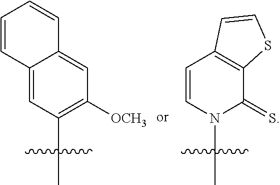 [SMILES: *c1cc2ccccc2cc1OC ; "or" ; *n1ccc2ccsc2c1=S]

51. The method of claim 44, wherein the aminoacyl tRNA synthetase is from Methanosarcina barkeri.

52. The method of claim 44, wherein the aminoacyl tRNA synthetase is from Methanococcus jannaschii.

53. The method of claim 44, wherein the cell is a microorganism or bacterium.

54. The method of claim 44, wherein the cell is E. coli.
